(19) United States
(12) Patent Application Publication
(10) Pub. No.: US 2011/0263484 A1
(43) Pub. Date: Oct. 27, 2011

(54) SINGLE CHAIN FC TYPE III INTERFERONS AND METHODS OF USING SAME

(75) Inventors: Margaret D. Moore, Seattle, WA (US); Michael G. Dodds, Normandy Park, WA (US); Paul O. Sheppard, Granite Falls, WA (US); Henrik Andersen, Seattle, WA (US)

(73) Assignee: ZymoGenetics, Inc.

(21) Appl. No.: 13/123,144

(22) PCT Filed: Oct. 13, 2009
(86) PCT No.: PCT/US09/60553

§ 371 Date: Jul. 11, 2011

Related U.S. Application Data
(60) Provisional application No. 61/116,601, filed on Nov. 20, 2008.
(60) Provisional application No. 61/104,946, filed on Oct. 13, 2008.

(51) Int. Cl. A61K 38/16; C07H 21/04; C12N 15/63; C12N 1/19; C12P 21/06; A61P 31/12; C12N 5/10; C07K 14/00; C12N 1/21

(52) U.S. Cl. 514/3.7; 530/350; 514/21.2; 536/23.1; 435/320.1; 435/252.33; 435/69.7; 435/252.31; 435/252.34; 435/252.3; 435/348; 435/254.2; 435/254.21; 435/254.23; 435/358; 435/352; 435/369; 435/350; 435/351; 435/364; 435/365; 435/354; 435/366; 435/353

(57) ABSTRACT

The present invention relates to single chain Fc Type III Interferon fusion proteins and methods of using same. The single chain Fc Type III Interferon fusion proteins comprise at least one Type III Interferon, two Fc regions and at least one linker, can be produced in a variety of single chain configurations, and are effector function minus or have a substantially reduced effector function.

### CROSS-REFERENCE TO RELATED APPLICATIONS

The present application is a 371 application of PCT/US2009/060553, filed Oct. 13, 2009, which claims priority benefit of U.S. Provisional Application Ser. Nos. 61/104,946, filed Oct. 13, 2008, and 61/116,601, filed Nov. 20, 2008, each of which is incorporated by reference herein.

### BACKGROUND OF THE INVENTION

It has been estimated that 3% of the world's population, i.e., 130 million individuals are infected with hepatitis C. Stauber R E and Stadlbauer V., Journal of Clinical Virology, 36:87-94 (2006). The majority have been infected via parenteral exposure with contaminated injections, either related to injection drug use or contaminated injections or transfusion with blood products received as part of an individual' health care. The current standard of care for hepatitis C is pegylated interferon (PEG-IFN) alpha (given once weekly) in combination with oral ribavirin (given daily). Heathcote J. and Main J., Journal of Viral Hepatitis, 12:223-235 (2005).

Chronic infection with hepatitis C virus (HCV) is a leading cause of cirrhosis, liver failure, and hepatocellular carcinoma in the United States and worldwide. The primary goal of treatment is to eradicate the virus and prevent development of long-term complications. Successful treatment is defined as achievement of a sustained virologic response (SVR) as evidenced by undetectable HCV RNA levels at least 6 months following discontinuation of therapy (Pearlman B L. Hepatitis C treatment update. Am J Med 2004; 117 (5):344-352).

For patients infected with genotype 1 HCV, the most common genotype in the United States, treatment consists of weekly administration of a PEGylated interferon alpha (PEG-IFN-α) in combination with daily ribavirin for 48 weeks. The two currently approved forms of PEG IFN-α are peginterferon alpha 2a (PEGASYS®), and peginterferon alpha-2b (PEG-INTRON®), both of which are associated with SVR rates of approximately 50% in patients infected with genotype 1 HCV (Seeff L B. Natural history of chronic hepatitis C. Hepatology 2002A; 36 (5 Suppl 1):S35-46; Strader D B, Wright T, Thomas D L, Seeff L B. Diagnosis, management, and treatment of hepatitis C. Hepatology 2004; 39 (4):1147-1171). For those patients who fail to achieve an SVR, there is currently no standard treatment.

Relapsed patients, who compose approximately 20% of all treated genotype 1 HCV patients, represent a unique population of PEG-IFN-α treatment failures (Hadziyannis S J, Sette H, Jr., Morgan T R, Balan V, Diago M, Marcellin P, Ramadori G, Bodenheimer H, Jr., Bernstein D, Rizzetto M, Zeuzem S, Pockros P J, Lin A, Ackrill A M. Peginterferon-alpha2a and ribavirin combination therapy in chronic hepatitis C: a randomized study of treatment duration and ribavirin dose. Ann Intern Med 2004; 140 (5):346-355). While these patients have undetectable HCV RNA levels at the end of treatment, they relapse with detectable HCV RNA levels less than 6 months later (Hoofnagle J H, Seeff L B. Peginterferon and ribavirin for chronic hepatitis C. N Engl J Med 2006; 355 (23):2444-2451). Factors contributing to relapse may include dose reduction in ribavirin, especially during the first 24 weeks of treatment (Shiffman M L. Chronic hepatitis C: treatment of pegylated interferon/ribavirin nonresponders. Curr Gastroenterol Rep 2006; 8 (1):46-52.). Upon retreatment with IFN α based therapy, relapsed patients may manifest decreases in HCV RNA levels similar to those seen during their prior course of therapy (Strader D B, Wright T, Thomas D L, Seeff L B. Diagnosis, management, and treatment of hepatitis C. Hepatology 2004; 39 (4):1147-1171), and in cases where prior therapy consisted of a non-PEGylated IFN-α, may be able to achieve an SVR with retreatment utilizing a PEG-IFN-α and ribavirin (Jacobson I M, et al., A randomized trial of pegylated interferon alpha-2b plus ribavirin in the retreatment of chronic hepatitis C. Am J Gastroenterol 2005; 100 (11):2453-2462; Mathew A, et al., Sustained viral response to pegylated interferon alpha-2b and ribavirin in chronic hepatitis C refractory to prior treatment. Dig Dis Sci 2006; 51 (11):1956-1961; Shiffman M L, Chronic hepatitis C: treatment of pegylated interferon/ribavirin nonresponders. Curr Gastroenterol Rep 2006; 8 (1):46-52). This pattern of failure and response to retreatment suggests that relapsed patients retain the potential to respond to interferon-based therapy and therefore are a unique population in which to study the potential effects of novel interferon-like molecules (Hoofnagle J H, Seeff L B. Peginterferon and ribavirin for chronic hepatitis C. N Engl J Med 2006; 355 (23):2444-2451; FDA CDER Antiviral Drugs Advisory Committee. Summary Minutes of the Antiviral Drugs Advisory Committee, Oct. 19-20, 2006).

Treatment with PEG-IFN-α and ribavirin is associated with significant side effects. Major toxicities of PEG-IFN-α include flu-like symptoms; hematologic abnormalities including neutropenia, thrombocytopenia, and anemia; and neuropsychiatric disorders, most commonly depression. Other toxicities include gastrointestinal disturbances and dermatologic, autoimmune, and cardiac conditions. Elevations in liver transaminases have also been reported, particularly with peginterferon alpha 2a (Gish R G. Treating hepatitis C: the state of the art. Gastroenterol Clin North Am 2004; 33 (1 Suppl):S1-9; Hoffmann-La Roche Inc. Package Insert: PEGASYS(R) (peginterferon alfa-2a). 2005B:1-46). Ribavirin is associated with a number of adverse effects, most notably hemolytic anemia, which in combination with the myelosuppressive effects of IFN-α can be a significant clinical problem (Kowdley K V. Hematologic side effects of interferon and ribavirin therapy. J Clin Gastroenterol 2005; 39 (1 Suppl):S3-8; Strader D B, Wright T, Thomas D L, Seeff L B. Diagnosis, management, and treatment of hepatitis C. Hepatology 2004; 39 (4):1147-1171).

The toxicities associated with PEG-IFN-α and ribavirin often lead to delays in starting therapy, as well as dose reductions and early discontinuation of treatment (Pearlman B L. Hepatitis C treatment update. Am J Med 2004; 117 (5):344-352), all of which decrease the likelihood of achieving SVR. Adherence to therapy (defined as receiving ≧80% of the prescribed PEG IFN-α dose and ≧80% of the ribavirin dose for the duration of therapy) has been associated with higher SVR rates in genotype 1 HCV patients (McHutchison J G, et al., Adherence to combination therapy enhances sustained response in genotype-1-infected patients with chronic hepatitis C. Gastroenterology 2002; 123 (4):1061-1069).

The Fc portion of an antibody molecule includes the CH2 and CH3 domains of the heavy chain and a portion of the hinge region. It was originally defined by digestion of an IgG molecule with papain. Fc is responsible for two of the highly desirable properties of an IgG: recruitment of effector function and a long serum half life. The ability to kill target cells to which an antibody is attached stems from the activation of immune effector pathway (ADCC) and the complement pathway (CDC) through binding of Fc to Fc receptors and the complement protein, C1q, respectively. The binding is mediated by residues located primarily in the lower hinge region and upper CH2 domain (Wines, et al., J. Immunol. (2000) 164, 5313; Woof and Burton, Nature Reviews (2004) 4, 1.). The long half life in serum demonstrated by IgG is mediated through a pH dependant interaction between amino acids in the CH2 and CH3 domains and the neonatal receptor, FcRn (Ghetie and Ward, Immunology Today (1997) 18, 592; Petkova, et al., Int. Immunol. (2006) 18, 1759).

Formation of a dimer, comprising two CH2-CH3 units, is required for the functions provided by intact Fc. Interchain disulfide bonds between cysteines in the hinge region help hold the two chains of the Fc molecule together to create a functional unit. However, even in the absence of the hinge region, the CH3 domains have a strong tendency to associate, leading to the formation of non-covalent dimers (Theis, et al. J. Mol. Biol. (1999) 293, 67; Chames and Baty, FEMS Micorobiol. Lett. (2000) 189, 1). The association between CH3 domains is random and largely independent of other structural domains to which they are attached. The random pairing of CH3 domains limits the types of scFc Type III Interferon fusion proteins that can be attached to the Fc and, unless the units attached to CH2-CH3 are identical, the product formed in a cell is a mixture of homodimers and heterodimers that are very difficult to separate biochemically.

Given the limitations of current therapy, there remains a need for improved treatments for HCV and other diseaeses. Thus, there remains a need in the art for Type III Interferons having a longer half-life that can be developed into potent therapeutics, while being effectively and efficiently produced at large-scale in any number of available production systems. The scFc Type III Interferon Fuson protein overcomes the problems in the art associated with dimerization of separate Fc monomers.

### BRIEF DESCRIPTION OF THE DRAWINGS

FIGS. 1A-1C illustrate the amino acid sequences of certain immunoglobulin Fc polypeptides. Amino acid sequence numbers are based on the EU index (Kabat et al., Sequences of Proteins of Immunological Interest, US Department of Health and Human Services, NIH, Bethesda, 1991). The illustrated sequences include a wild-type human sequence (“wt”; SEQ ID NO:15) and four variant sequences, Fc4 (SEQ ID NO:11), Fc5 (SEQ ID NO:12), Fc6 (SEQ ID NO:13), and Fc7 (SEQ ID NO:14). The Cys residues normally involved in disulfide bonding to the light chain constant region (LC) and heavy chain constant region (HC) are indicated. A “.” indicates identity to wild-type at that position. *** indicates the stop codon; the C-terminal Lys residue has been removed from Fc6. Boundaries of the hinge, CH2, and CH3 domains are shown.

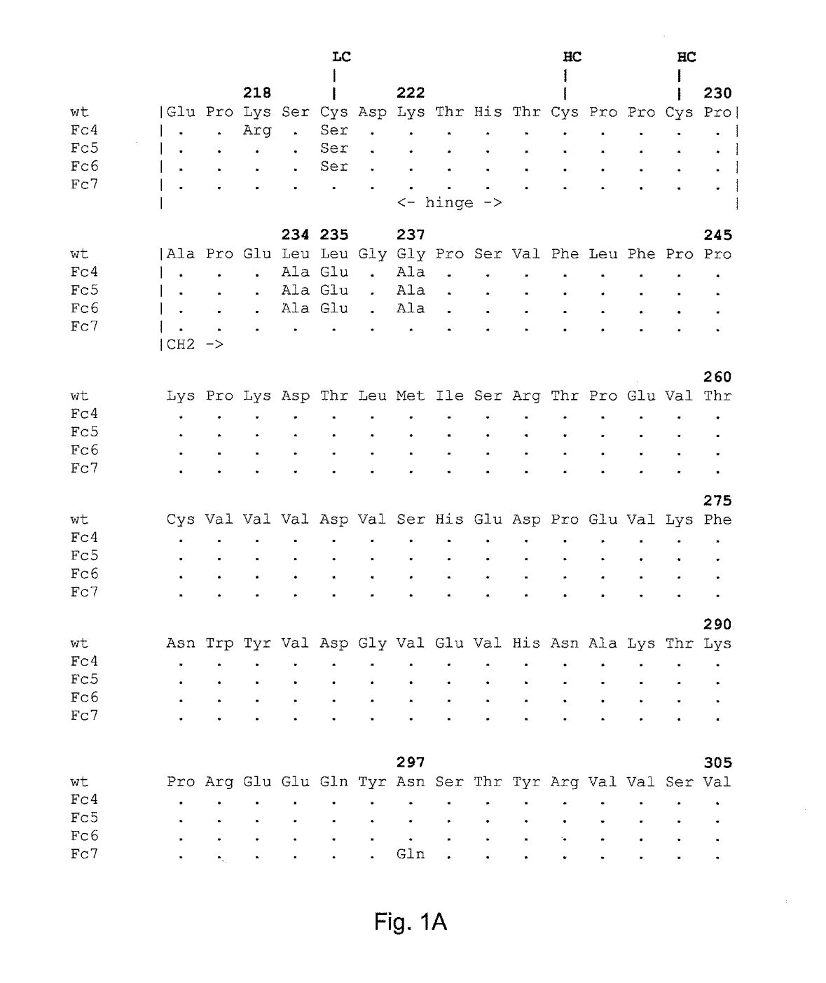

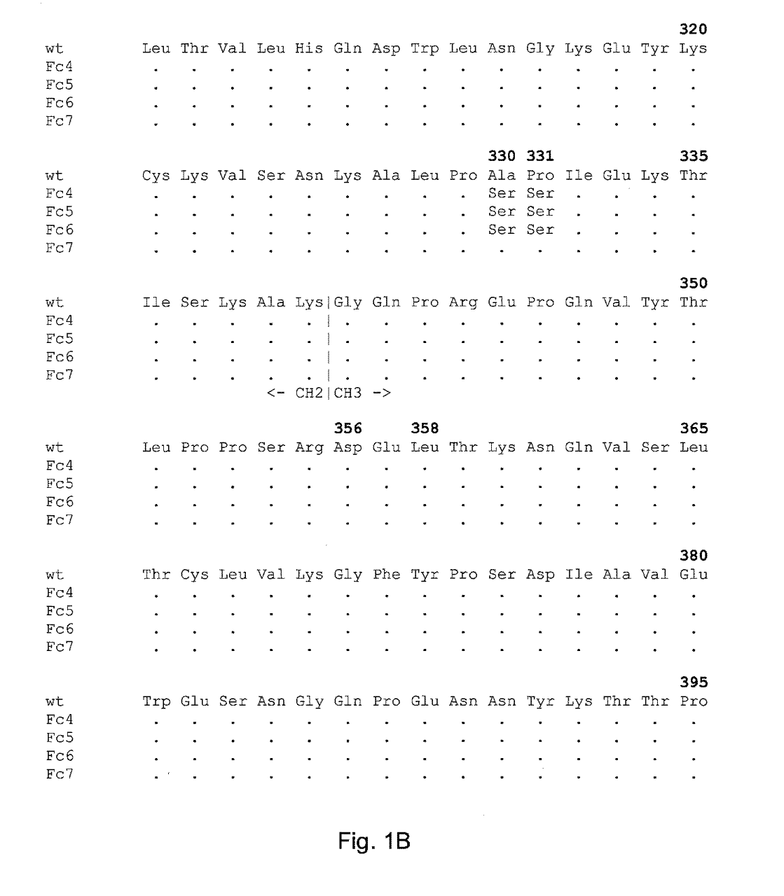

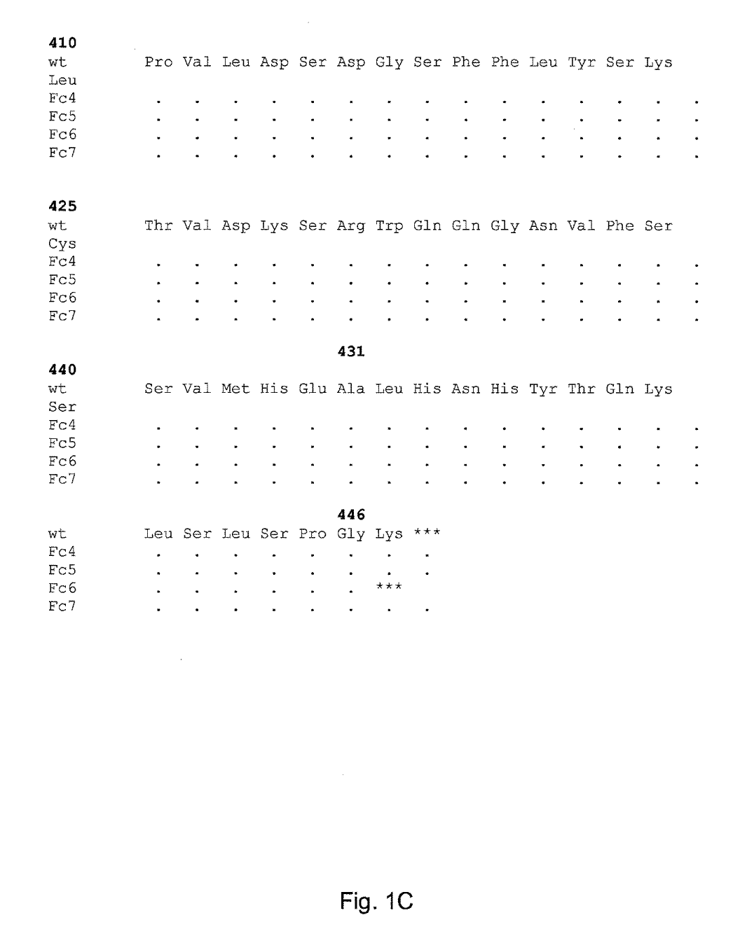

### DETAILED DESCRIPTION OF THE INVENTION

### Definitions

In the description that follows, a number of terms are used extensively. The following definitions are provided to facilitate understanding of the invention.

The terms “a,” “an,” and “the” include plural referents, unless the context clearly indicates otherwise.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

A “bivalent molecule” is a fusion protein comprising two Type III Interferons.

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

As used herein, the term “Fc portion” or “Fc monomer” means a polypeptide comprising at least one CH2 domain and one CH3 domain of an immunoglobulin molecule. An Fc monomer can be a polypeptide comprising at least a fragment of the constant region of an immuno globulin excluding the first constant region immunoglobulin domain of the heavy chain (CH1), but maintaining at least part of one CH2 domain and one CH3 domain, wherein the CH2 domain is amino terminal to the CH3 domain. In one aspect of this definition, an Fc monomer can be a polypeptide constant region comprising a portion of the hinge region, a CH2 region and a CH3 region. Such Fc polypeptide molecules can be obtained by papain digestion of an immunoglobulin region, for example and not limitation. In another aspect of this definition, an Fc monomer can be a polypeptide region comprising a portion of a CH2 region and a CH3 region. Such Fc polypeptide molecules can be obtained by pepsin digestion of an immunoglobulin molecule, for example and not limitation. In one embodiment, the polypeptide sequence of an Fc monomer is substantially similar to an Fc polypeptide sequence of an IgG1 Fc region, an IgG2 Fc region, an IgG3 Fc region, an IgG4 Fc region, an IgM Fc region, an IgA Fc region, an IgD Fc region and an IgE Fc region. (See, e.g., Padlan, Molecular Immunology, 31 (3), 169-217 (1993)). Because there is some variation between immunoglobulins, and solely for clarity, Fc monomer refers to the last two constant region immunoglobulin domains of IgA, IgD, and IgG, and the last three constant region immunoglobulin domains of IgE and IgM. As mentioned, the Fc monomer can also include the flexible hinge N-terminal to these domains. For IgA and IgM, the Fc monomer may include the J chain. For IgG, the Fc portion comprises immunoglobulin domains CH2 and CH3 and the hinge between CH1 and CH2. Although the boundaries of the Fc portion may vary, the human IgG heavy chain Fc portion is usually defined to comprise residues C226 or P230 to its carboxyl-terminus, wherein the numbering is according to the EU index as in Kabat. The Fc portion may refer to this region in isolation, or this region in the context of an Fc polypeptide, as described below. By “Fc polypeptide” as used herein is meant a polypeptide that comprises all or part of an Fc monomer. Fc polypeptides include antibodies, Fc fusions, isolated Fc molecules, functional Fc fragments and functional variants thereof.

A “fusion protein” or a “fusion polypeptide” is a hybrid protein or polypeptide expressed by a nucleic acid molecule comprising nucleotide sequences of at least two genes of portions thereof. For example, a fusion protein can comprise at least part of a Fc domain fused with a Type III Interferon polypeptide.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical foams, such as dimers or alternatively glycosylated or derivatized forms.

The terms “nucleic acid” or “nucleic acid molecule” refer to a deoxyribonucleotide or ribonucleotide polymer in either single-or double-stranded form, and unless otherwise limited, would encompass known analogs of natural nucleotides that can function in a similar manner as naturally occurring nucleotides. A “nucleotide sequence” also refers to a polynucleotide molecule or oligonucleotide molecule in the form of a separate fragment or as a component of a larger nucleic acid. The nucleotide sequence or molecule may also be referred to as a “probe” or a “primer.” Some of the nucleic acid molecules of the invention are derived from DNA or RNA isolated at least once in substantially pure form and in a quantity or concentration enabling identification, manipulation, and recovery of its component nucleotide sequence by standard biochemical methods. Examples of such methods, including methods for PCR protocols that may be used herein, are disclosed in Sambrook et al., Molecular Cloning: A Laboratory Manual, 2nd ed., Cold Spring Harbor Laboratory Press, New York (1989), Ausubel, F. A., et al., eds., Current Protocols in Molecular Biology, John Wiley and Sons, Inc., New York (1987), and Innis, M., et al., (Eds.) PCR Protocols: A Guide to Methods and Applications, Academic Press, San Diego, Calif. (1990). Reference to a nucleic acid molecule also includes its complement as determined by the standard Watson-Crick base-pairing rules, with uracil (U) in RNA replacing thymine (T) in DNA, unless the complement is specifically excluded. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like.

As described herein, the nucleic acid molecules of the invention include DNA in both single-stranded and double-stranded form, as well as the DNA or RNA complement thereof. DNA includes, for example, DNA, genomic DNA, chemically synthesized DNA, DNA amplified by PCR, and combinations thereof. Genomic DNA, including translated, non-translated and control regions, may be isolated by conventional techniques, e.g., using any one of the cDNAs of the invention, or suitable fragments thereof, as a probe, to identify a piece of genomic DNA which can then be cloned using methods commonly known in the art.

A “nucleic acid molecule construct” is a nucleic acid molecule, either single-stranded or double-stranded, that has been modified through human intervention to contain segments of nucleic acid combined and juxtaposed in an arrangement not existing in nature.

The term “operably linked”, when referring to DNA segments, indicates that the segments are arranged so that they function in concert for their intended purposes, e.g., transcription initiates in the promoter and proceeds through the coding segment to the terminator.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired.

A “polypeptide” is a polymer of amino acid residues joined by peptide bonds, whether produced naturally or synthetically. Polypeptides of less than about 10 amino acid residues are commonly referred to as “peptides”.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-peptide structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

A “recombinant host” is a cell that contains a heterologous nucleic acid molecule, such as a cloning vector or expression vector. In the present context, an example of a recombinant host is a cell that produces a multispecific antibody or antibody fragment of the present invention from an expression vector.

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

As used herein, the terms “single-chain Fc,” “scFc” “scFc polypeptide” or “scFc molecule” are used interchangeably and refer to a molecule comprising at least two Fc portions within a single polypeptide chain.

A “therapeutically effective amount” of a composition is that amount that produces a statistically significant effect, such as a statistically significant reduction in disease progression or a statistically significant improvement in organ function. The exact dose will be determined by the clinician according to accepted standards, taking into account the nature and severity of the condition to be treated, patient traits, etc. Determination of dose is within the level of ordinary skill in the art.

A polypeptide “variant” as referred to herein means a polypeptide substantially homologous to a native polypeptide, but which has an amino acid sequence different from that encoded by any of the nucleic acid sequences of the invention because of one or more deletions, insertions or substitutions. Amino acid sequence insertions include amino- and/or carboxyl-terminal fusions ranging in length from one residue to polypeptides containing a hundred or more residues, as well as intrasequence insertions of single or multiple amino acid residues. Intrasequence insertions (e.g., insertions within the target polypeptide sequence) may range generally from about 1 to 10 residues, more preferably 1 to 5, most preferably 1 to 3. Variants can comprise conservatively substituted sequences, meaning that a given amino acid residue is replaced by a residue having similar physiochemical characteristics. See, Zubay, Biochemistry, Addison-Wesley Pub. Co., (1983). It is a well-established principle of protein and peptide chemistry that certain amino acids substitutions, entitled “conservative” amino acid substitutions, can frequently be made in a protein or a peptide without altering either the confirmation or the function of the protein or peptide. Such changes include substituting any of isoleucine (I), valine (V), and leucine (L) for any other of these amino acids; aspartic acid (D) for glutamic acid (E) and vice versa; glutamine (Q) for asparagine (N) and vice versa; and serine (S) for threonine (T) and vice versa. Ordinarily, variants will have an amino acid sequence having at least 75% amino acid sequence identity with the reference sequence, more preferably at least 80%, more preferably at least 85%, more preferably at least 90%, and most preferably at least 95%. Identity or homology with respect to this sequence is defined herein as the percentage of amino acid residues in the candidate sequence that are identical with the reference sequence residues, after aligning the sequences and introducing gaps, if necessary, to achieve the maximum percent sequence identity. Preferably, variants will retain the primary function of the parent from which it they are derived.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

The above-mentioned substitutions are not the only amino acid substitutions that can be considered “conservative.” Other substitutions can also be considered conservative, depending on the environment of the particular amino acid. For example, glycine (G) and alanine (A) can frequently be interchangeable, as can be alanine and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments. The effects of such substitutions can be calculated using substitution score matrices such PAM120, PAM-200, and PAM-250 as discussed in Altschul, (J. Mol. Biol. 219:55565 (1991)). Other such conservative substitutions, for example, substitutions of entire regions having similar hydrophobicity characteristics, are well known.

Naturally-occurring peptide variants are also encompassed by the invention. Examples of such variants are proteins that result from alternate mRNA splicing events or from proteolytic cleavage of the polypeptides described herein. Variations attributable to proteolysis include, for example, differences in the N- or C-termini upon expression in different types of host cells, due to proteolytic removal of one or more terminal amino acids from the polypeptides encoded by the sequences of the invention.

The interferon lambdas are a newly described family of cytokines, related to both type-1 interferons and IL-10 family members. The family, classified as the “Type III” Interferons, is comprised of three novel four helical bundle cytokines designed as IFN-λ2.1, IFN-λ2 and IFN-λ3 (also referred to as IL-29 or zcyto21, IL-28A or zcyto20, and IL-28B or zcyto22, respectively. Jordan W J et al., Genes and Immunity, 8:13-20 (2007). All three interferons lambdas signal through a heterodimeric receptor complex composed of the class II cytokine receptors IL-28RA (also known as IL-28 receptor alpha) and CRF2-4 (also known as IL-10RB or IL-10R2. The IL-28 receptor is quite distinct from that used by Type I Interferons.

IFN-λ1 or IL-29 is a member of the recently described Type III interferon family (Kotenko S V et al., IFN-lambdas mediate antiviral protection through a distinct class II cytokine receptor complex. Nat Immunol 2003; 4 (1):69-77; Sheppard P et al., IL-28, M-29 and their class II cytokine receptor IL-28R. Nat Immunol 2003; 4 (1):63-68) with functional similarities to Type I interferons, which include IFN-α and IFN-β (Ank, et al., 2006). Similarly to IFN-α, the Type III interferons are induced in response to viral infection and stimulates an intracellular response that involves phosphorylation of signal transducing activator of transcription (STAT) proteins and induction of interferon-responsive genes, also known as interferon stimulated genes (ISGs). ISGs encode proteins involved in antiviral responses and immune stimulation, including Protein kinase R (PkR), Myxovirus resistance (Mx), 2′5′ oligoadenylate synthetase (OAS), and β2-microglobulin (B2M) (Samuel C E. Antiviral actions of interferons. Clin Microbiol Rev 2001; 14 (4):778-809; Stark G R, Kerr I M, Williams B R, Silverman R H, Schreiber R D. How cells respond to interferons. Annu Rev Biochem 1998; 67:227-264).

The Type III Interferon polynucleotide and polypeptide sequences of the present invention are those Type III Interferon sequences known by one of skill in the art and can be found, for instance, in U.S. Pat. Nos. 6,927,040, 7,038,032, 7,135,170, 7,157,559, 7,253,261, 7,351,689, 7,241,870, and in U.S. Patent Publication Nos. 20070020227, 20070053933, 20080096252, 20080075693, all of which are incorporated herein by reference in their entirety.

Additional IL-29 polypeptides of the present invention include various modifications of the IL-29 base sequence (SEQ ID NO:1). These additional IL-29 polypeptides of the present invention include, for example, SEQ ID NOs:17, 19, 21, 23 and 25, which are encoded by IL-29 polynucleotide molecules as shown in SEQ ID NOs:16, 18, 20, 22 and 24, respectively. The IL-29 polypeptides of the present invention include an amino-terminus truncated IL-29 polypeptide, denoted IL-29 N1 (SEQ ID NO:17—amino acid residues 1-18 of SEQ ID NO:1 are deleted, and the cysteines at amino acid positions 94 and 153 of SEQ ID NO:1 are substituted with a serine); a carboxy-terminus truncated IL-29 polypeptide, denoted IL-29 C1 (SEQ ID NO:19—amino acid residues 1-6 and 168-181 of SEQ ID NO:1 are deleted); a further carboxy-terminus truncated IL-29 polypeptide, denoted IL-29 C2 (SEQ ID NO:21—amino acid residues 1-6 and 164-181 of SEQ ID NO:1 are deleted); an N1 amino-terminus truncated IL-29 polypeptide and a C1 carboxy-terminus truncated IL-29 polypeptide, denoted as IL-29 N1C1 (SEQ ID NO:23—amino acid residues 1-18 and 168-181 of SEQ ID NO:1 are deleted, and the cysteine at amino acid residue 94 is substituted with a serine); and an N1 amino-terminus truncated IL-29 polypeptide and a C2 carboxy-terminus truncated IL-29 polypeptide, denoted as IL-29 N1C2 (SEQ ID NO:25—amino acid residues 1-18 and 164-181 of SEQ ID NO:1 are deleted, and the cysteine at amino acid residue 94 is substituted with a serine).

Expression of the receptor for the Type III interferons is more restricted than that of the IFN-α receptor. For example, while all cell types in the liver express the IFN-α receptor, the receptor for the Type III interferons is found only in hepatocytes. Similarly, in peripheral blood, high levels of the receptor for the Type III interferons are detected only on B cells, whereas all peripheral blood leukocytes (PBLs) including B, T, and NK cells, neutrophils, and monocytes express the IFN-α receptor. Consistent with this pattern of receptor expression, treatment of PBLs with the Type III interferons leads to low levels of STAT-1 phosphorylation in B cells but not in other PBLs. This is in contrast to IFN-α, which induces STAT 1 phosphorylation in all PBLs tested.

The present invention provides for an isolated fusion protein comprising from the amino-terminus to the carboxy-terminus a Type III Interferon polypeptide, a linker polypeptide and a scFc polypeptide comprising at least two Fc monomers and at least one linker. The Type III Interferon polypeptides are IL-28A, IL-28B or IL-29. One example, for instance, of an IL-29 polypeptide of the present invention is shown in SEQ ID NOs:1, 17, 19, 21, 23 and 25. One example, for instance, of an IL-28A polypeptide of the present invention is shown in SEQ ID NO:2. One example, for instance, of an IL-28B polypeptide of the present invention is shown in SEQ ID NO:3. The IL-29 portion of the fusion protein can have at least 90% or 95% sequence identity to amino acid residues 1 to 181 of SEQ ID NO:1 or, for instance, any other IL-29 sequence incorporated herein by reference. The IL-28A portion of the fusion protein can have at least 90% or 95% sequence identity to amino acid residues 1 to 175 of SEQ ID NO:2 or, for instance, any other IL-28A sequence incorporated herein by reference. The IL-28B portion of the fusion protein can have at least 90% or 95% sequence identity to amino acid residues 1 to 175 of SEQ ID NO:3 or, for instance, any other IL-28B sequence incorporated herein by reference. The linker polypeptide of the fusion protein can be a Gly-Ser linker with the following formula (Gly4Ser)n, wherein n is 1-10. Optionally, the linker polypeptide can be a linker selected from the group consisting of SEQ ID NO:4, SEQ ID NO:5, SEQ ID NO:6, SEQ 1D NO:7, SEQ ID NO:8, SEQ ID NO:9 and SEQ ID NO:10. The scFc polypeptide of the fusion protein comprises a first Fc monomer comprising a CH2 domain and a CH3 domain and a second Fc monomer comprising a CH2 domain and a CH3 domain. Optionally, the first Fc monomer and said second Fc monomer are arranged in an amino to carboxyl order selected from: a) Hinge-CH2-CH3-linker-Hinge-CH2-CH3; b) Hinge-CH2-CH3-linker-CH2-CH3; c) Hinge-CH2-linker-Hinge-CH2-CH3-linker-CH3; d) Hinge-CH2-linker-CH2-CH3-linker-CH3; e) linker-CH2-CH3-linker-CH2-CH3; and CH2-linker-CH2-CH3-linker-CH3. The first and second Fc monomers or the scFc polypeptide can be effector function minus or have a substantially reduced effector function. The first and second Fc monomers can be, for example, SEQ ID NO:11, SEQ ID NO:12, SEQ ID NO:13 or SEQ ID NO:14. The present invention may further comprise from the carboxy-terminus of the scFc polypeptide a second linker polypeptide and a second Type III Interferon polypeptide. The second linker polypeptide of the fusion protein can be a Gly-Ser linker with the following formula (Gly₄Ser)n, wherein n is 1 to 10. Optionally, the second linker polypeptide can be a linker selected from the group consisting of SEQ ID NO:4, SEQ ID NO:5, SEQ ID NO:6, SEQ ID NO:7, SEQ ID NO:8, SEQ ID NO:9 and SEQ ID NO:10.

An additional embodiment of the present invention provides that the above isolated fusion protein further comprise from the amino-terminus of the Type III Interferon polypeptide or from the carboxy-terminus of the second Type III Interferon polypeptide a third linker polypeptide and a third Type III Interferon polypeptide. Optionally, the third linker polypeptide is (Gly₄Ser)ₙ, wherein n is 1-10, such as SEQ ID NO:4, SEQ ID NO:5, SEQ ID NO:6, SEQ ID NO:7, SEQ ID NO:8, SEQ ID NO:9 or SEQ ID NO:10. Optionally, the third Type III Interferon polypeptide is IL-28A, IL-28B or IL-29. The IL-28A polypeptide may have at least 90% or 95% sequence identity to amino acid residues 1 to 175 of SEQ ID NO:2. The IL-28B polypeptide may have at least 90% or 95% sequence identity to amino acid residues 1 to 175 of SEQ ID NO:3. The EL-29 polypeptide may have at least 90% or 95% sequence identity to amino acid residues 1 to 181 of SEQ ID NO:1, 1 to 163 of SEQ ID NO:17, 1 to 161 of SEQ ID NO:19, 1 to 157 of SEQ ID NO:21, 1 to 149 of SEQ ID NO:23 or 1 to 145 of SEQ ID NO:25. Optionally, the fusion protein may further comprise from the amino-terminus of the Type III Interferon polypeptide a fourth linker polypeptide and a fourth Type III Interferon polypeptide. Optionally, the fourth linker polypeptide is (Gly₄Ser)ₙ, wherein n is 1-10, such as SEQ ID NO:4, SEQ ID NO:5, SEQ ID NO:6, SEQ ID NO:7, SEQ ID NO:8, SEQ ID NO:9 or SEQ ID NO:10. Optionally, the fourth Type III Interferon polypeptide is IL-28A, IL-28B or IL-29. The IL-28A polypeptide may have at least 90% or 95% sequence identity to amino acid residues 1 to 175 of SEQ ID NO:2. The IL-28B polypeptide may have at least 90% or 95% sequence identity to amino acid residues 1 to 175 of SEQ ID NO:3. The IL-29 polypeptide may have at least 90% or 95% sequence identity to amino acid residues 1 to 181 of SEQ ID NO:1, 1 to 163 of SEQ ID NO:17, 1 to 161 of SEQ ID NO:19, 1 to 157 of SEQ ID NO:21, 1 to 149 of SEQ ID NO:23 or 1 to 145 of SEQ ID NO:25. Optionally, the isolated fusion protein further comprises from the carboxy-terminus of the second Type III Interferon polypeptide a fourth linker polypeptide and a fourth Type III Interferon polypeptide. Optionally, the fourth linker polypeptide is (Gly₄Ser)ₙ, wherein n is 1-10, such as SEQ ID NO:4, SEQ ID NO:5, SEQ ID NO:6, SEQ ID NO:7, SEQ ID NO:8, SEQ ID NO:9 or SEQ ID NO:10. Optionally, the fourth Type III Interferon polypeptide is IL-28A, IL-28B or IL-29. The IL-28A polypeptide may have at least 90% or 95% sequence identity to amino acid residues 1 to 175 of SEQ ID NO:2. The IL-28B polypeptide may have at least 90% or 95% sequence identity to amino acid residues 1 to 175 of SEQ ID NO:3. The IL-29 polypeptide may have at least 90% or 95% sequence identity to amino acid residues 1 to 181 of SEQ ED NO:1, 1 to 163 of SEQ ID NO:17, 1 to 161 of SEQ ID NO:19, 1 to 157 of SEQ ID NO:21, 1 to 149 of SEQ ID NO:23 or 1 to 145 of SEQ ID NO:25.

Another embodiment of the present invention provides for an isolated fusion protein comprising from the amino-terminus to the carboxy-terminus a scFc polypeptide comprising at least two Fc monomers and at least one linker, a linker polypeptide, and a Type III Interferon polypeptide. Optionally, the Type III Interferon polypeptide is IL-28A, IL-28B or IL-29. The IL-28A polypeptide may have at least 90% or 95% sequence identity to amino acid residues 1 to 175 of SEQ ID NO:2. The IL-28B polypeptide may have at least 90% or 95% sequence identity to amino acid residues 1 to 175 of SEQ ID NO:3. The IL-29 polypeptide may have at least 90% or 95% sequence identity to amino acid residues 1 to 181 of SEQ ID NO:1, 1 to 163 of SEQ ID NO:17, 1 to 161 of SEQ ID NO:19, 1 to 157 of SEQ ID NO:21, 1 to 149 of SEQ ID NO:23 or 1 to 145 of SEQ ID NO:25. Optionally, the linker polypeptide is (Gly₄Ser)ₙ, wherein n is 1-10, such as SEQ ID NO:4, SEQ ID NO:5, SEQ ID NO:6, SEQ ID NO:7, SEQ ID NO:8, SEQ ID NO:9 or SEQ ID NO:10. Optionally, the scFc polypeptide comprises a first Fc monomer comprising a CH2 domain and a CH3 domain and a second Fc monomer comprising a CH2 domain and a CH3 domain. Optionally, the first Fc monomer and said second Fc monomer are arranged in an amino to carboxyl order selected from: a) Hinge-CH2-CH3-linker-Hinge-CH2-CH3; b) Hinge-CH2-CH3-linker-CH2-CH3; c) Hinge-CH2-linker-Hinge-CH2-CH3-linker-CH3; d) Hinge-CH2-linker-CH2-CH3-linker-CH3; e) linker-CH2-CH3-linker-CH2-CH3; and f)CH2-linker-CH2-CH3-linker-CH3. Optionally, the first and second Fc monomers have no effector function or have a substantially reduced effector function, such as SEQ ID NO:11, SEQ ID NO:12, SEQ ID NO:13 and/or SEQ ID NO:14. Optionally, the isolated fusion protein further comprises from the amino-terminus of the scFc polypeptide a second linker polypeptide and a second Type III Interferon polypeptide. Optionally, the second linker polypeptide is (Gly₄Ser)ₙ, wherein n is 1-10, such as SEQ ID NO:4, SEQ ID NO:5, SEQ ID NO:6, SEQ ID NO:7, SEQ ID NO:8, SEQ ID NO:9 or SEQ ID NO:10.

Another embodiment of the present invention provides for an isolated fusion protein comprising from the amino-terminus to the carboxy-terminus a scFc polypeptide comprising at least two Fc monomers and at least one linker, a linker polypeptide, and a Type III Interferon polypeptide. Optionally, the Type III Interferon polypeptide is IL-28A, IL-28B or IL-29. The IL-28A polypeptide can have at least 90% or 95% sequence identity to amino acid residues 1 to 175 of SEQ ID NO:2. The IL-28B polypeptide can have at least 90% or 95% sequence identity to amino acid residues 1 to 175 of SEQ ID NO:3. The IL-29 polypeptide can have at least 90% or 95% sequence identity to amino acid residues 1 to 181 of SEQ ID NO:1, 1 to 163 of SEQ ID NO:17, 1 to 161 of SEQ ID NO:19, 1 to 157 of SEQ ID NO:21, 1 to 149 of SEQ ID NO:23 or 1 to 145 of SEQ ID NO:25. Optionally, the linker polypeptide is (Gly₄Ser)ₙ, wherein n is 1-10, such as for instance, SEQ ID NO:4, SEQ ID NO:5, SEQ ID NO:6, SEQ ID NO:7, SEQ ID NO:8, SEQ ID NO:9 and SEQ ID NO:10. Optionally, the scFc polypeptide comprises a first Fc monomer comprising a CH2 domain and a CH3 domain and a second Fc monomer comprising a CH2 domain and a CH3 domain Optionally, the first Fc monomer and said second Fc monomer are arranged in an amino to carboxyl order selected from: a) Hinge-CH2-CH3-linker-Hinge-CH2-CH3; b) Hinge-CH2-CH3-linker-CH2-CH3; c) Hinge-CH2-linker-Hinge-CH2-CH3-linker-CH3; d) Hinge-CH2-linker-CH2-CH3-linker-CH3; e) linker-CH2-CH3-linker-CH2-CH3; and f) CH2-linker-CH2-CH3-linker-CH3. Optionally, the first and second Fc monomers have no effector function or have a substantially reduced effector function, such as SEQ ID NO:11, SEQ ID NO:12, SEQ ID NO:13 or SEQ ID NO:14. The isolated fusion protein may further comprise from the amino-terminus of the scFc polypeptide a second linker polypeptide and a second Type III Interferon polypeptide. Optionally, the second linker polypeptide is (Gly₄Ser)ₙ, wherein n is 1-10, such as SEQ ID NO:4, SEQ ID NO:5, SEQ ID NO:6, SEQ ID NO:7, SEQ ID NO:8, SEQ ID NO:9 and SEQ ID NO:10.

The present invention also provides for a composition comprising a fusion protein as described herein and a pharmaceutically acceptable carrier.

The present invention also provides for an isolated polynucleotide encoding a fusion protein as described herein.

The present invention also provides for an expression vector comprising the following operably linked elements: (a) transcription promoter; (b) a DNA segment encoding a fusion protein as described herein; and (c) transcription terminator.

The present invention also provides for a cultured cell comprising an expression vector as described herein.

The present invention also provides for a method of producing a fusion protein comprising culturing a cell as described herein under conditions wherein the fusion protein is expressed from an expression vector as described herein. The method further comprises recovering the expressed fusion protein.

The present invention also provides for a method of treating a patient having a viral infection comprising administering to the patient a therapeutically effective amount of a composition as described herein, wherein after administration of the composition the viral load has reduced or viral replication is inhibited. Optionally, the viral infection is from a virus selected from the group consisting of DNA Viruses (e.g., Herpes Viruses such as Herpes Simplex viruses, Epstein-Barr virus, Cytomegalovirus; Pox viruses such as Variola (small pox) virus; Hepadnaviruses (e.g., Hepatitis B virus); Papilloma viruses; Adenoviruses); RNA Viruses (e.g., HIV I, II; HTLV I, II; Poliovirus; Hepatitis A; Orthomyxoviruses (e.g., Influenza viruses); Paramyxoviruses (e.g., Measles virus); Rabies virus; Hepatitis C); Rhinovirus, Respiratory Syncytial Virus, West Nile Virus, Yellow Fever, Rift Valley Virus, Lassa Fever Virus, Ebola Virus, Lymphocytic Choriomeningitis Virus, Human Immunodeficiency virus, viral meningitis, severe acute respiratory syndrome (SARS) coronavirus and HIV-related disease. Optionally, the patient has a Hepatitis C infection.

The present invention also provides for a method of treating a patient having an autoimmune disorder comprising administering to the patient a therapeutically effective amount of a composition as described herein, wherein the autoimmune disorder to selected from the group consisting of multiple sclerosis, arthritis, rheumatoid arthritis, inflammatory bowel disease, systemic lupus erythematosus, and psoriasis.

The present invention also provides for a method of treating a patient having cancer comprising administering to the patient a therapeutically effective amount of a composition as described herein, wherein the type of cancer is selected from the group consisting of lymphoproliferative disorders, including for instance, B-cell lymphomas, chronic lymphocytic leukemia, acute lymphocytic leukemia, Non-Hodgkin's lymphomas, multiple myeloma, acute myelocytic leukemia, chronic myelocytic leukemia, renal cell carcinoma, cervical cancer (e.g., squamous type and adenocarcinoma), head and neck tumours (e.g., Hypopharyngeal Cancer, Laryngeal Cancer, Lip and Oral Cavity Cancer, Metastatic Squamous Neck Cancer with Occult Primary, Nasopharyngeal Cancer, Oropharyngeal Cancer, Paranasal Sinus and Nasal Cavity Cancer, Parathyroid Cancer, and Salivary Gland Cancer), melanoma (e.g., malignant melanoma such as Superficial spreading melanoma, Nodular melanoma, and Lentigo maligna melanoma), thyroid carcinoma (e.g., Papillary, Follicular, Medullary, and Anaplastic), malignant gliomas (e.g., gliobastoma multiforme and anaplastic astrocytoma), breast cancer (e.g., ductal carcinoma), colon cancer, lung cancer (e.g., small cell lung cancer, non-small cell lung cancer such as Squamous cell carcinoma, Adenocarcinoma and Large cell carcinoma, and mesothelioma), pancreatic cancer, prostate cancer, stomach cancer, ovarian cancer, testicular cancer, Kaposi's sarcoma, and bone cancer (e.g., Osteosarcoma, Ewing's sarcoma, Chondrosarcoma, Spindle cell sarcoma, and Chordoma).

As discussed above, the Fc portion of an antibody comprises the CH2 and CH3 domains of an immunoglobulin molecule. The propensity of the hinge and CH3 domains of an antibody to associate and the proximity associated within a single chain construct make it possible for two Fc portions connected by a polypeptide linker and one or more Type III Interferons to fold properly. Thus the scFc molecule produces molecule with improved half-life. Additional information on single chain Fc polypeptides can be found at U.S. patent application Ser. No. 12/106,081, which is herein incorporated by reference in its entirety.

Fc4 (Effector function minus .gamma.1 Fc with BglII site; SEQ ID NO:11). Arg 218 was introduced in the hinge region to include a BglII restriction enzyme recognition sequence to facilitate cloning. Cys 220 is the Cys residue that forms the disulfide bond to the light chain constant region in an intact immunoglobulin IgG1 protein. Since the Fc fusion protein constructs do not have a light chain partner, Fc4 includes a Ser for Cys residue substitution to prevent deleterious effects due to the potential presence of an unpaired sulfhydral group. In the CH2 region three amino acid substitutions were introduced to reduce Fc.gamma.receptorI (Fc.gamma.RI) binding. These are the substitutions at EU index positions 234, 235, and 237. These substitutions were described by Greg Winter's group in Duncan et al., Nature 332:563 (1988) and were shown in that paper to reduce binding to the Fc.gamma.RI.

Two amino acid substitutions in the complement C1q binding site were introduced to reduce complement fixation. These are the substitutions at EU index positions 330 and 331. The importance, or relevance, of positions 330 and 331 in complement C1q binding (or lack of complement fixation or activation) is described by Sherie Morrison's group in Tao et al., J. Exp. Med. 178:661 (1993) and Canfield and Morrison, J. Exp. Med. 173:1483 (1991). The CH3 region in the Fc4 variant remains identical to the wild type .gamma.1 Fc.

Fc5 (Effector function minus .gamma.1 Fc without the BglII site; SEQ ID NO:12) Fc5 is a variant of Fc4. In the Fc5 hinge region the Arg 218 substitution was returned to the wild type Lys 218 residue. Fc5 contains the same Cys 220 to Ser substitution as described above for Fc4. Fc5 contains the same CH2 substitutions as does Fc4, and the Fc5 CH2 region is identical to the wild type .gamma.1 Fc.

Fc6 (Effector function minus .gamma.1 Fc without the BglII site and lacking the C-terminal Lys residue; SEQ ID NO:13). The Fc6 variant contains the same hinge region substitutions as Fc5 and contains the same CH2 substitutions as Fc4. The Fc6 CH3 region does not contain a carboxyl terminal lysine residue. This particular Lys residue does not have an assigned EU index number. This lysine is removed to a varying degree from mature immunoglobulins and therefore predominantly not found on circulating antibodies. The absence of this residue on recombinant Fc fusion proteins may result in a more homogeneous product.

Fc7 (Aglycosylated .gamma.1 Fc; SEQ ID NO:14). The Fc7 variant is identical to the wild type .gamma.1 Fc in the hinge region. In the CH2 region the N-linked carbohydrate attachment site at residue Asn-297 is changed to Gin to produce a deglycosylated Fc. (See e.g., Tao and Morrison (1989) J. Immunol. 143:2595-2601). The CH3 region is identical to the wild type .gamma.1 Fc.

Other Fc variants are possible, including without limitation one in which a region capable of forming a disulfide bond is deleted, or in which certain amino acid residues are eliminated at the N-terminal end of a native Fc form or a methionine residue is added thereto. Thus, in one embodiment of the invention, one or more Fc portions of the scFc molecule can comprise one or more mutations in the hinge region to eliminate disulfide bonding. In yet another embodiment, the hinge region of an Fc can be removed entirely. In still another embodiment, the scFc molecule can comprise an Fc variant.

Further, an Fc variant can be constructed to remove or substantially reduce effector functions by substituting, deleting or adding amino acid residues to effect complement binding or Fc receptor binding. For example, and not limitation, a deletion may occur in a complement-binding site, such as a C1q-binding site. Techniques of preparing such sequence derivatives of the immunoglobulin Fc fragment are disclosed in International Patent Publication Nos. WO 97/34631 and WO 96/32478. In addition, the Fc domain may be modified by phosphorylation, sulfation, acrylation, glycosylation, methylation, farnesylation, acetylation, amidation, and the like.

The Fc may be in the form of having native sugar chains, increased sugar chains compared to a native form or decreased sugar chains compared to the native form, or may be in an aglycosylated or deglycosylated form. The increase, decrease, removal or other modification of the sugar chains may be achieved by methods common in the art, such as a chemical method, an enzymatic method or by expressing it in a genetically engineered production cell line. Such cell lines can include microorganisms, e.g. Pichia Pastoris, and mammalians cell line, e.g. CHO cells, that naturally express glycosylating enzymes. Further, microorganisms or cells can be engineered to express glycosylating enzymes, or can be rendered unable to express glycosylation enzymes (See e.g., Hamilton, et al., Science, 313:1441 (2006); Kanda, et al, J. Biotechnology, 130:300 (2007); Kitagawa, et al., J. Biol. Chem., 269 (27): 17872 (1994); Ujita-Lee et al., J. Biol. Chem., 264 (23): 13848 (1989); Imai-Nishiya, et al, BMC Biotechnology 7:84 (2007); and WO 07/055916). As one example of a cell engineered to have altered sialylation activity, the alpha-2,6-sialyltransferase 1 gene has been engineered into Chinese Hamster Ovary cells and into sf9 cells. Antibodies expressed by these engineered cells are thus sialylated by the exogenous gene product. A further method for obtaining Fc molecules having a modified amount of sugar residues compared to a plurality of native molecules includes separating said plurality of molecules into glycosylated and non-glycosylated fractions, for example, using lectin affinity chromatography (See e.g., WO 07/117505). The presence of particular glycosylation moieties has been shown to alter the function of Immunoglobulins. For example, the removal of sugar chains from an Fc molecule results in a sharp decrease in binding affinity to the C1q part of the first complement component C1 and a decrease or loss in antibody-dependent cell-mediated cytotoxicity (ADCC) or complement-dependent cytotoxicity (CDC), thereby not inducing unnecessary immune responses in vivo. Additional important modifications include sialylation and fucosylation: the presence of sialic acid in IgG has been correlated with anti-inflammatory activity (See e.g., Kaneko, et al, Science 313:760 (2006)), whereas removal of fucose from the IgG leads to enhanced ADCC activity (See e.g., Shoj-Hosaka, et al, J. Biochem., 140:777 (2006)).

The CH2 and CH3 domains may be derived from humans or other animals including cows, goats, swine, mice, rabbits, hamsters, rats and guinea pigs, and preferably humans, or synthetic, or a combination thereof. In addition, the Fc portion may be derived from IgG, IgA, IgD, IgE and IgM, or that is made by combinations thereof or hybrids thereof. More specifically, the scFc molecules of the present invention are based on the joining of two Fc portions by a linker to form a multimer.

The linkers can be naturally-occurring, synthetic or a combination of both. For example, a synthetic linker can be a randomized linker, e.g., both in sequence and size. In one aspect, the randomized linker can comprise a fully randomized sequence, or optionally, the randomized linker can be based on natural linker sequences. The linker can comprise, e.g, a non-polypeptide moiety, a polynucleotide, a polypeptide or the like. A linker can be rigid, or alternatively, flexible, or a combination of both. Linker flexibility can be a function of the composition of both the linker and the subunits that the linker interacts with. A suitable length is, e.g., a length of at least one and typically fewer than about 50 amino acid residues, such as 2-25 amino acid residues, 5-20 amino acid residues, 5-15 amino acid residues, 8-12 amino acid residues or 11 residues. Other suitable polypeptide linker sizes may include, e.g., from about 2 to about 15 amino acids, from about 3 to about 15, from about 4 to about 12, about 10, about 8, or about 6 amino acids. The amino acid residues selected for inclusion in the linker polypeptide should exhibit properties that do not interfere significantly with the activity or function of the polypeptide multimer. Thus, the peptide linker should, on the whole, not exhibit a charge that would be inconsistent with the activity or function of the linked polypeptides, or interfere with internal folding, or form bonds or other interactions with amino acid residues in one or more of the domains that would seriously impede the linked polypeptides in question. Preferred linkers include polypeptide linkers such as (Gly₄Ser)ₙ, wherein n is 1 to 10.

The linker can also be a non-peptide linker, such as a non-peptide polymer. The term “non-peptide polymer”, as used herein, refers to a biocompatible polymer including two or more repeating units linked to each other by a covalent bond excluding the peptide bond. Examples of the non-peptide polymer include poly (ethylene glycol), poly (propylene glycol), copolymers of ethylene glycol and propylene glycol, polyoxyethylated polyols, polyvinyl alcohol, polysaccharides, dextran, polyvinyl ether, biodegradable polymers such as PLA (poly (lactic acid) and PLGA (poly (lactic-glycolic acid), lipid polymers, chitins, and hyaluronic acid. The most preferred is poly (ethylene glycol) (PEG).

In one embodiment, linkers are used to join two Fc monomers to form an scFc molecule. A linker can also be used to join one or more selected Type III Interferons to an scFc polypeptide. Configurations of molecules comprising an scFc and optionally comprising one or more Type III Interferons are described herein. Linkers to join polypeptide fragments are generally known in the art and can be used to form scFc molecules in accordance of the present invention. Linkers allow the separate, discrete domains to cooperate yet maintain their separate properties. In some cases, a disulfide bridge exists between two linked Type III Interferons or between a linker and a Type III Interferon.

Choosing a suitable linker for an scFc Type III Interferon fusion protein comprising one or more Type III Interferons may depend on a variety of parameters including, e.g., the nature of the Fc domains being linked, the nature of any one or more Type III Interferons, the structure and nature of the target to which the composition should bind, and/or the stability of the linker (e.g., peptide linker) towards proteolysis and oxidation.

Particularly suitable linker polypeptides predominantly include amino acid residues selected from Glycine (Gly), Serine (Ser), Alanine (Ala), and Threonine (Thr). For example, the peptide linker may contain at least 75% (calculated on the basis of the total number of residues present in the peptide linker), such as at least 80%, at least 85%, or at least 90% of amino acid residues selected from Gly, Ser, Ala, and Thr. The peptide linker may also consist of Gly, Ser, Ala and/or Thr residues only. The linker polypeptide should have a length that is adequate to link two Fc monomers, and optionally, one or more Type III Interferons to an scFc polypeptide or to each other in such a way that the linked regions assume the correct conformation relative to one another so that they retain the desired activity.

One example where the use of peptide linkers is widespread is for production of single-chain antibodies where the variable regions of a light chain (VL) and a heavy chain (VH) are joined through an artificial linker, and a large number of publications exist within this particular field. A widely used peptide linker is a 15mer consisting of three repeats of a Gly-Gly-Gly-Gly-Ser amino acid sequence ((Gly₄Ser)₃) (SEQ ID NO:4). Other linkers have been used, and phage display technology, as well as selective infective phage technology, has been used to diversify and select appropriate linker sequences (Tang et al., J. Biol. Chem. 271, 15682-15686, 1996; Hennecke et al., Protein Eng. 11, 405-410, 1998). Peptide linkers have been used to connect individual chains in hetero- and homo-dimeric proteins such as the T-cell receptor, the lambda Cro repressor, the P22 phage Arc repressor, IL-12, TSH, FSH, IL-5, and interferon-.gamma. Peptide linkers have also been used to create fusion polypeptides. Various linkers have been used, and, in the case of the Arc repressor, phage display has been used to optimize the linker length and composition for increased stability of the single-chain protein (See Robinson and Sauer, Proc. Natl. Acad. Sci. USA 95, 5929-5934, 1998).

Still another way of obtaining a suitable linker is by optimizing a simple linker (e.g., (Gly₄Ser)ₙ) through random mutagenesis.

As stated, a linker can be rigid, or flexible, or a combination of both. Linker flexibility can be a function of the composition of both the linker and the Type III Interferons that the linker interacts with (e.g., the scFv polypeptides, or Fc domains). The linker joins two Fc monomers, one or more Type III Interferons to the scFc polypeptide. Thus the linker can allow the separate discrete Fc monomers and/or Type III Interferons to remain connected in a way that each binding entity binds its target. In one embodiment, it is generally preferred that the peptide linker possess at least some flexibility. Accordingly, in some variations, the peptide linker contains 1-25 glycine residues, 5-20 glycine residues, 5-15 glycine residues, or 8-12 glycine residues. Particularly suitable peptide linkers typically contain at least 50% glycine residues, such as at least 75% glycine residues. In some embodiments, a peptide linker comprises glycine residues only.

In certain variations, the peptide linker comprises other residues in addition to the glycine. Preferred residues in addition to glycine include Ser, Ala, and Thr, particularly Ser. One example of a specific peptide linker includes a peptide linker having the amino acid sequence GlyₓXaa-Gly_{y}-Xaa-Gly_{z} (SEQ ID NO:5), wherein each Xaa is independently selected from Alanine (Ala), Valine (Val), Leucine (Leu), Isoleucine (Ile), Methionine (Met), Phenylalanine (Phe), Tryptophan (Trp), Proline (Pro), Glycine (Gly), Serine (Ser), Threonine (Thr), Cysteine (Cys), Tyrosine (Tyr), Asparagine (Asn), Glutamine (Gln), Lysine (Lys), Arginine (Arg), Histidine (His), Aspartate (Asp), and Glutamate (Glu), and wherein x, y, and z are each integers in the range from 1-5. In some embodiments, each Xaa is independently selected from the group consisting of Ser, Ala, and Thr. In a specific variation, each of x, y, and z is equal to 3 (thereby yielding a peptide linker having the amino acid sequence Gly-Gly-Gly-Xaa-Gly-Gly-Gly-Xaa-Gly-Gly-Gly (SEQ ID NO:6), wherein each Xaa is selected as above).

In some cases, it may be desirable or necessary to provide some rigidity into the peptide linker. This may be accomplished by including proline residues in the amino acid sequence of the peptide linker Thus, in another embodiment, a peptide linker comprises at least one proline residue in the amino acid sequence of the peptide linker. For example, a peptide linker can have an amino acid sequence wherein at least 25% (e.g., at least 50% or at least 75%) of the amino acid residues are proline residues. In one particular embodiment of the invention, the peptide linker comprises proline residues only.

In certain variations, a peptide linker comprises at least one cysteine residue, such as one cysteine residue. For example, in some embodiments, a peptide linker comprises at least one cysteine residue and amino acid residues selected from the group consisting of Gly, Ser, Ala, and Thr. In some such embodiments, a peptide linker comprises glycine residues and cysteine residues, such as glycine residues and cysteine residues only. Typically, only one cysteine residue will be included per peptide linker. One example of a specific peptide linker comprising a cysteine residue includes a peptide linker having the amino acid sequence Gly-Cys-Glyₙ (SEQ ID NO:7), wherein n and m are each integers from 1-12, e.g., from 3-9, from 4-8, or from 4-7. In a specific variation, such a peptide linker has the amino acid sequence GGGGG-C-GGGGG (SEQ ID NO:8).

The linkers used to join the Fc monomers of an scFc polypeptide may be positioned between the CH3 of a first Fc monomer and the CH2 of a second Fc monomer. More specifically, a single chain construct can be designed such that a linker may be placed between any of the following: CH2-CH2, CH2-CH3, CH3-CH3, CH2-CH3 and CH2-CH3, CH2-CH2 and CH3-CH3, CH2-hinge region, CH3-hinge region, CH3 of a first Fc monomer—CH2 of a second Fc monomer, and CH2 of a first Fc monomer—CH3 of a second Fc monomer, as long as the scFc polypeptide forms the a desired structure. This scFc can then also be combined with one to improve stability. The scFc polypeptide is described with reference to Fc molecules having two constant regions.

The scFc polypeptides of the present invention include variants having single or multiple amino acid substitutions, deletions, additions, or replacements that do not retain or have substantially reduced the biological properties (e.g., effector function) of the molecules of the invention. Thus, the present invention encompasses effector function minus or substantially reduced effector function single chain Fc Type III Interferon fusion proteins in which the scFc polylpeptide thereof comprises Fc portions that are based on amino acid sequence variants of the native Fc polypeptide sequences. These variants are prepared by introducing appropriate nucleotide changes into the DNA encoding the Fc or by in vitro synthesis of the desired Fc. Such variants include, for example, humanized variants of non-human Fc domains, as well as deletions from, or insertions or substitutions of, residues within particular amino acid sequences of an Fc domain. Any combination of deletion, insertion, and substitution can be made to arrive at the final construct, provided that the final construct possesses the desired characteristics. The amino acid changes also may alter post-translational processing of the target polypeptide, such as changing the number or position of glycosylation sites, introducing a membrane anchoring sequence into the constant domain or modifying the leader sequence of the native Fc.

DNA encoding the amino acid sequence variants of the scFc Type III Interferons fusion proteins of the present invention is prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the target polypeptide or by total gene synthesis. These techniques may utilize target polypeptide nucleic acid (DNA or RNA), or nucleic acid complementary to the target polypeptide nucleic acid. Oligonucleotide-mediated mutagenesis is a preferred method for preparing substitution, deletion, and insertion variants of target polypeptide DNA.

The cDNA or genomic DNA encoding the single chain Fc Type III Interferon fusion proteins can be inserted into a replicable vector for further cloning (amplification of the DNA) or for expression. Many vectors are available, and selection of the appropriate vector will depend on 1) whether it is to be used for DNA amplification or for expression of the encoded protein, 2) the size of the DNA to be inserted into the vector, and 3) the host cell to be transformed with the vector. Each vector contains various components depending on its function (amplification of DNA or expression of DNA) end the host cell for which it is compatible. The vector components generally include, but are not limited to, one or more of the following: a signal sequence, an origin of replication, one or more marker genes, a promoter, and a transcription termination sequence.

In general, the signal sequence may be a component of the vector, or it may be a part of the target polypeptide DNA that is inserted into the vector. Included within the scope of this invention are Type III Interferons with any native signal sequence deleted and replaced with a heterologous signal sequence. The heterologous signal sequence selected should be one that is recognized and processed (e.g., cleaved by a signal peptidase) by the host cell. For prokaryotic host cells that do not recognize and process the native polypeptide signal sequence, the signal sequence is substituted by a prokaryotic signal sequence selected, for example, from the group of the alkaline phosphatase, penicillinase, Ipp, or heat-stable enterotoxin II leaders.

Expression and cloning vectors may, but need not, contain a polynucleotide sequence that enables the scFc Type III Interferon fusion polynucleotide to replicate in one or more selected host cells. Generally, in cloning vectors this sequence is one that enables the vector to replicate independently of the host chromosomal DNA, and includes origins of replication or autonomously replicating sequences. Such sequences are well known for a variety of microbes. The origin of replication from the plasmid pBR322 is suitable for most Gram-negative bacteria.

DNA may also be replicated by insertion into the host genome. This is readily accomplished using Bacillus species as hosts, for example, by including in the vector a DNA sequence that is complementary to a sequence found in Bacillus genomic DNA. Transfection of Bacillus with this vector results in homologous recombination with the genome and insertion of the target polypeptide DNA. However, the recovery of genomic DNA encoding the binding molecule polypeptide is more complex than that of an exogenously replicated vector because restriction enzyme digestion is required to excise the target polypeptide DNA. Similarly, DNA also can be inserted into the genome of vertebrate and mammalian cells by conventional methods.

Expression and cloning vectors should contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g. ampicillin, neomycin, methotrexate, or tetracycline, (b) complement auxotrophic deficiencies; or (c) supply critical nutrients not available from complex media, e.g. the gene encoding D-alanine racemase for Bacilli.

Expression and cloning vectors will usually contain a promoter that is recognized by the host organism and is operably linked to the scFc Type III Interferon fusion nucleic acid. Promoters are untranslated sequences located upstream (5′) to the start codon of a structural gene (generally within about 100 to 1000 bp) that control its transcription and translation. Such promoters typically fall into two classes, inducible and constitutive. Inducible promoters are promoters that initiate increased levels of transcription from DNA under their control in response to some change in culture conditions, e.g. the presence or absence of a nutrient or a change in temperature.

Construction of suitable vectors containing one or more of the above listed components employs standard ligation techniques. Isolated plasmids or DNA fragments are cleaved, tailored, and relegated in the form desired to generate the plasmids required.

Suitable host cells for expressing single chain Fc Type III Interferon fusion protein of the present invention are microbial cells such as yeast, fungi, insect and prokaryotes. Suitable prokaryotes include eubacteria, such as Gram-negative or Gram-positive organisms, for example, E. coli, Bacilli such as B. subtilis, Pseudomonas species such as P. aeruginosa, Salmonella typhimurium, or Serratia marcescans. One preferred E. coli cloning host is E. coli 294 (American Type Cell Culture, Manassas, Va. ATCC 31,446), although other strains such as E. coli B, E. coli .sub.X 1776 (ATCC 31,537), E. coli RV308 (ATCC 31,608) and E. coli W3110 (ATCC 27,325) are suitable.

Host cells of the invention also include any insect expression cell line known, such as for example, Spodoptera frugiperda cells.

The expression cell lines may also be yeast cell lines, such as, for example, Saccharomyces cerevisiae, Pichia pastoris, Pichia methanolia and Schizosaccharomyces pombe cells.

The expression cells may also be mammalian cells such as, for example, hybridoma cells (e.g., NS0 cells), Chinese hamster ovary cells (CHO), baby hamster kidney cells, human embryonic kidney line 293, normal dog kidney cell lines, normal cat kidney cell lines, monkey kidney cells, African green monkey kidney cells, COS cells, and non-tumorigenic mouse myoblast G8 cells, fibroblast cell lines, myeloma cell lines, mouse NIH/3T3 cells, LMTK31 cells, mouse sertoli cells, human cervical carcinoma cells, buffalo rat liver cells, human lung cells, human liver cells, mouse mammary tumor cells, TRI cells, MRC 5 cells, and FS4 cells.

Expression cells may be engineered to provide an exogenous cellular activity or to remove an endogenous cellular activity. One non-limiting example includes the addition of a sialyltransferase gene to a cell to increase the sialylation of molecules expressed therefrom. Thus, such a cell can then be further manipulated to express a scFc Type III Interferon fusion protein of the current invention and said cell will express a sialylated scFc Type III Interferon fusion protein. In one embodiment, a CHO cell line is engineered to include express exogenous 2,6-sialyltransferase gene and to further express an scFc molecule of the current invention. Expression cells may be cultured in the presence of agents that modulate the cell's endogenous protein production and/or activity. In one example, a cell can be cultured in an altered cell culture process that includes one or more of: adding an alkanoic acid; altering the osmolarity or altering the cell culture temperature to control the amount of sialylic acid that the cell adds to a glycoprotein produced in the host cell. See e.g., U.S. Pat. No. 5,705,364.

These examples are illustrative rather than limiting. Preferably the host cell should secrete minimal amounts of proteolytic enzymes, and additional protease inhibitors may desirably be incorporated in the cell culture.

Host cells are transfected and preferably transformed with the above-described expression or cloning vectors of this invention and cultured in conventional nutrient media modified as appropriate for inducing promoters, selecting transformants, or amplifying the genes encoding the desired sequences.

Cells used to produce the scFc Type III Interferon fusion proteins of the present invention are cultured in suitable media as described generally in Sambrook et al., (Molecular Cloning: A Laboratory Manual New York: Cold Spring Harbor Laboratory Press, 1989). Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

It is currently preferred that the bacterial host cells be cultured at temperatures from 37° C. to 29° C., although temperatures as low as 20° C. may be suitable. Optimal temperatures will depend on the host cells, the Fc sequence and other parameters. 37 ° C. is generally preferred.

Methods of purification are known in the art. In some embodiments of the invention, methods for purification include filtration, affinity column chromatography, cation exchange chromatography, anion exchange chromatography, and concentration. In general, soluble binding molecule polypeptides are recovered from recombinant cell culture to obtain preparations that are substantially homogeneous. As a first step, the culture medium or periplasmic preparation is centrifuged to remove particulate cell debris. Periplasmic preparations are obtained in conventional fashion, e.g. by freeze-thaw or osmotic shock methods. The membrane and soluble protein fractions are then separated. The scFc Type III Interferon fusion protein is then purified from the soluble protein fraction. The following procedures are exemplary of suitable purification procedures: fractionation on immunoaffinity or ion-exchange columns; ethanol precipitation; reverse phase HPLC; chromatography on silica or on a cation exchange resin such as DEAE; chromatofocusing; SDS-PAGE; ammonium sulfate precipitation; gel filtration using, for example, Sephadex G-75; protein A or protein G affinity matrix (e.g. Sepharose) columns; and hydrophobic interaction chromotography. More specifically, the filtration step preferably comprises ultrafiltration, and more preferably ultrafiltration and diafiltration. Filtration is preferably performed at least about 5-50 times, more preferably 10 to 30 times, and most preferably 14 to 27 times. Affinity column chromatography, may be performed using, for example, PROSEP Affinity Chromatography (Millipore, Billerica, Mass.). In a one embodiment, the affinity chromatography step comprises PROSEP-VA column chromatography. Eluate may be washed in a solvent detergent. Cation exchange chromatography may include, for example, SP-Sepharose Cation Exchange Chromatography. Anion exchange chromatography may include, for example but not limited to, Q-Sepharose Fast Flow Anion Exchange. The anion exchange step is preferably non-binding, thereby allowing removal of contaminants including DNA and BSA. The scFc Type III Interferon fusion protein can be nanofiltered, for example, using a Pall DV 20 Nanofilter. The scFc Type III Interferon fusion protein may be concentrated, for example, using ultrafiltration and diafiltration. The method may further comprise a step of size exclusion chromatography to remove aggregates. Sialylated Fc fractions can be isolated using affinity chromatography with immobilized Sambucus nigra lectin (Vector labs), followed by elution with lactose (See e.g., Shibuya, et al, Archives of Biochemistry and Biophysics, 254 (1): 1 (1987)).

### Uses of the Single Chain Fc Type III Interferons

A. Antiviral Treatment

The present invention also provides for treating a patient having a viral infection comprising administering to the patient a therapeutically effective amount of a fusion protein of the present invention, wherein after administration of the fusion protein the viral load has reduced or viral replication is inhibited. Optionally, the the viral infection is a result of a virus selected from the group consisting of DNA Viruses (e.g., Herpes Viruses such as Herpes Simplex viruses, Epstein-Barr virus, Cytomegalovirus; Pox viruses such as Variola (small pox) virus; Hepadnaviruses (e.g, Hepatitis B virus); Papilloma viruses; Adenoviruses); RNA Viruses (e.g., HIV I, II; HTLV I, II; Poliovirus; Hepatitis A; Orthomyxoviruses (e.g., Influenza viruses); Paramyxoviruses (e.g., Measles virus); Rabies virus; Hepatitis C); Rhinovirus, Respiratory Syncytial Virus, West Nile Virus, Yellow Fever, Rift Valley Virus, Lassa Fever Virus, Ebola Virus, Lymphocytic Choriomeningitis Virus, Human Immunodeficiency virus, viral meningitis, severe acute respiratory syndrome (SARS) coronavirus and HIV-related disease. Optionally, the patient has an Hepatitis C infection.

In another embodiment, the treatment for a hepatitis C patient may further include, in addition to the scFc Type III Interferon fusion protein, at least one anti-hepatitis C agent. Optionally, the anti-hepatitis C agent is selected from the group consisting of polymerase and/or protease inhibitors, A3AR agonists, Toll-Like Receptor agonists, monoclonal antibodies, Botanicals, anti-phospholipids, immunomodulators, anti-inflammatory drugs, thiazolides, broad spectrum immune stimulators, inflammatory/fibrosis inhibitors, cyclophilin inhibitors, pancaspase inhibitors, HCV immune globulins, antivirals, anti-infectives, RNA inhibitiors, glucosidase I inhibitors, IRES inhibitors, bezafibrates, nucleoside analogs, Type I Interferons and Type II Interferons. The polymerase and/or protease inhibitor can be, for example, VCH-916 (Virochem), GS9190 (Gilead), GSK625433 (GlaxcoSmithKline), ITMN-191 (R-7227; InterMune), R7128 (Pharmasset/Roche), VCH-759 (Virochem), R1626 (Roche), TMC435350 (Medivir/Tibotec), SCH503034 (Boceprevir, Schering-Plough), A-831 (Arrow Therapeutics), valopicitabine (NM283, Idenix Pharmaceuticals) or VX950 (Telaprevir, Vertex). The A3AR agonist can be, for example, CF102 (Can-Fite). The Toll-Like Receptor agonist can be, for example, IMO-2125 (Idera Pharmaceuticals), Isatoribine (ANA971, Anadys Pharmaceuticals) or Actilon (CPG10101, Coley Pharmaceutical Group). The monoclonal antibody can be, for example, AB68 (XTL bio). The Botanical can be, for example, PYN17 (Phynova). The anti-phospholipid can be, for example, Bavituximab (formerly Tarvacin; Peregrine). The immunomodulator can be, for example, NOV-205 (Novelos Therapeutics), Oglufanide disodium (Implicit Bioscience) or thymalfasin (thymosin alpha 1; SciClone/Sigma-Tau). The anti-inflammatory drug can be, for example, CTS-1027 (Conatus) or JBK-122 (Jenken Biosciences). The thiazolides can be, for example, Alinia (nitazoxanide; Romark Laboratories). The broad spectrum immune stimulator can be, for example, SCV-07 (SciClone). The inflammatory/fibrosis inhibitor can be, for example, MitoQ (mitoquinone; Antipodean Pharmaceuticals). The cyclophilin inhibitor can be, for example, DEBIO-025 (Debio Pharm Group). The pancaspase inhibitor can be, for example, PF-03491390 (formerly IDN-6556; Pfizer Pharmaceuticals). The HCV immune globulin can be, for example, Civacir (Nabi). The antiviral can be, for example, Suvus (Methylene blue, formerly BIVN-104 (Virostat); Bioenvision). Optionally, the anti-infective is Nitazoxanide (Alinia®, Romark Pharmaceuticals). The glucosidase I inhibitor can be, for example, MX-3253 (celgosivir; Migenix). The IRES inhibitor can be, for example, VGX-410C (Mifepristone; VGX Pharmaceuticals). The bezafibrate can be, for example, Hepaconda (Giaconda). The nucleoside analog can be, for example, ribavirin (Roches's Copegus or Schering-Plough's Rebetol) or viramidine (taribavirin (ribavirin pro-drug); Valeant Pharmaceuticals). Optionally, the ribavirin or viramidine is administered orally once or twice daily to the patient at a dose of about 800-1200 mg. The Type I Interferon can be, for example, Interferon alpha or pegylated Interferon alpha. Optionally, the Interferon alpha or pegylated Interferon alpha is PEGASYS (pegylated interferon-alpha-2a or peg-IFN-α-2a; Roche), PEG-INTRON (pegylated interferon-alpha-2b or peg-IFN-α-2b; Schering-Plough), Belerofon (Nautilus Biotech), oral interferon alpha (Amarillo Biosciences), BLX-883 (Locteron; Biolex Therapeutics/OctoPlus), Multiferon (Viragen), Albuferon (Human Genome Sciences), Consensus Interferon or (Infergen; Three Rivers Pharma). The Type I Interferon can be, for example, omega interferon (Intarcia Therapeutics). Optionally, the Type II Interferon is Interferon gamma, e.g., Actimmune® by Intermune.

There are several in vivo models for testing HBV and HCV that are known to those skilled in art. With respect to HCV, for example, the HCV Replicon model is a cell-based system to study the effectiveness of a drug to inhibit HCV replication (Blight et al., Science, 290 (5498):1972-1974 (Dec. 8, 2000); and Lohmann et al., Science, 285 (5424):110-113 (Jul. 2, 1999)). A well-known and accepted in vitro HBV model to one of skill in the art can be used to determine the anti-HBV activity of a test molecule is disclosed in Korba et al., Antiviral Res., 19 (1):55-70 (1992) and Korba et al., Antiviral Res., 15 (3):217-228 (1991).

Suitable dosages for any of the above coadministered agents are those presently used and may be lowered due to the combined action (synergy) of the anti-hepatitis C agent and the scFc Type III Interferon fusion protein.

B. Cancer Treatment

An scFc Type III Interferon fusion protein can be used to treat any of the following disorders: carcinoma, a sarcoma, a glioma, a lymphoma, a leukemia, or a skin cancer. The carcinoma can be a skin, an esophageal, a gastric, a colonic, a rectal, a pancreatic, a lung, a breast, an ovarian, a urinary bladder, an endometrial, a cervical, a testicular, a renal, an adrenal or a liver carcinoma. B-cell related disease may be an indolent form of B-cell lymphoma, an aggressive form of B-cell lymphoma, non-Hodgkin's lymphoma, a chronic lymphocytic leukemia, an acute lymphocytic leukemia, a Waldenstrom's macroglobulinemia, or a multiple myeloma. In addition, the B-cell related disease can be a human or a veterinary type of disease. Neovascular disorders amenable to treatment in accordance with the present invention include, for example, cancers characterized by solid tumor growth (e.g., pancreatic cancer, renal cell carcinoma (RCC), colorectal cancer, non-small cell lung cancer (NSCLC), and gastrointestinal stromal tumor (GIST)) as well as various neovascular ocular disorders (e.g., age-related macular degeneration, diabetic retinopathy, iris neovascularization, and neovascular glaucoma). A T-cell related disease may be a human or veterinary T-cell leukemia, skin psoriasis, psoriatic arthritis or mycosis fungoides. A metabolic disease can be an amyloidosis. A neurodegenerative disease can be an Alzheimer's disease.

### 1. Types of Cancer

TABLE 1
Exemplary Cancers Involving Solid Tumor Formation

| 1. | Head and Neck cancer | |
|---|---|---|
| | a. | Brain |
| | b. | Oral cavity |
| | c. | Orophyarynx |
| | d. | Nasopharynx |
| | e. | Hypopharynx |
| | f. | Nasal cavities and paranasal sinuses |
| | g. | Larynx |
| | h. | Lip |
| 2. | Lung cancers | |
| | a. | Non-small cell carcinoma |
| | b. | Small cell carcinoma |
| 3. | Gastrointestinal Tract cancers | |
| | a. | Colorectal cancer |
| | b. | Gastric cancer |
| | c. | Esophageal cancer |
| | d. | Anal cancer |
| | e. | Extrahepatic Bile Duct cancer |
| | f. | Cancer of the Ampulla of Vater |
| | g. | Gastrointestinal Stromal Tumor (GIST) |
| 4. | Liver cancer | |
| | a. | Liver Cell Adenoma |
| | b. | Hepatocellular Carcinoma |
| 5. | Breast cancer | |
| 6. | Gynecologic cancer | |
| | a. | Cervical cancer |
| | b. | Ovarian cancer |
| | c. | Vaginal cancer |
| | d. | Vulvar cancer |
| | e. | Gestational Trophoblastic Neoplasia |
| | f. | Uterine cancer |
| 7. | Urinary Tract cancer | |
| | a. | Renal cancer carcinoma |
| | b. | Prostate cancer |
| | c. | Urinary Bladder cancer |
| | d. | Penile cancer |
| | e. | Urethral cancer |
| 8. | Urinary Bladder cancer | |
| 9. | Neurological Tumors | |
| | a. | Astrocytoma and glioblastoma |
| | b. | Primary CNS lymphoma |
| | c. | Medulloblastoma |
| | d. | Germ Cell tumors |
| | e. | Retinoblastoma |
| 10. | Endocrine Neoplasms | |
| | a. | Thyroid cancer |
| | b. | Pancreatic cancer |

| | |
|---|---|
| 1) | Islet Cell tumors |

| | |
|---|---|
| a) | Insulinomas |
| b) | Glucagonomas |

| | c. | Pheochromocytoma |
|---|---|---|
| | d. | Adrenal carcinoma |
| | e. | Carcinoid tumors |
| | f. | Parathyroid cancinoma |
| | g. | Pineal gland neoplasms |
| 11. | Skin cancers | |
| | a. | Malignant melanoma |
| | b. | Squamous Cell carcinoma |
| | c. | Basal Cell carcinoma |
| | d. | Kaposi's Sarcoma |
| 12. | Bone cancers | |
| | a. | Osteoblastoma |
| | b. | Osteochondroma |
| | c. | Osteosarcoma |
| 13. | Connective Tissue neoplasms | |
| | a. | Chondroblastoma |
| | b. | Chondroma |
| 14. | Hematopoietic malignancies | |
| | a. | Non-Hodgkin Lymphoma |

| | |
|---|---|
| 1) | B-cell lymphoma |
| 2) | T-cell lymphoma |
| 3) | Undifferentiated lymphoma |

| | |
|---|---|
| b. | Leukemias |

| | |
|---|---|
| 1) | Chronic Myelogenous Leukemia |
| 2) | Hairy Cell Leukemia |
| 3) | Chronic Lymphocytic Leukemia |
| 4) | Chronic Myelomonocytic Leukemia |
| 5) | Acute Myelocytic Leukemia |
| 6) | Acute Lymphoblastic Leukemia |

| | |
|---|---|
| c. | Myeloproliferative Disorders |

| | |
|---|---|
| 1) | Multiple Myeloma |
| 2) | Essential Thrombocythemia |
| 3) | Myelofibrosis with Myeloid Metaplasia |
| 4) | Hypereosinophilic Syndrome |
| 5) | Chronic Eosinophilic Leukemia |
| 6) | Polycythemia Vera |

| | d. | Hodgkin Lymphoma |
|---|---|---|
| 15. | Childhood Cancers | |
| | a. | Leukemia and Lymphomas |
| | b. | Brain cancers |
| | c. | Neuroblastoma |
| | d. | Wilm's Tumor (nephroblastoma) |
| | e. | Phabdomyosarcoma |
| | f. | Retinoblastoma |
| 16. | Immunotherapeutically sensitive cancers | |
| | a. | melanoma |
| | b. | kidney cancer |
| | c. | leukemias, lymphomas and myelomas |
| | d. | breast cancer |
| | e. | prostate cancer |
| | f. | colorectal cancer |
| | g. | cervical cancer |
| | h. | ovarian cancer |
| | i. | lung cancer |

Some of the cancers listed above, including some of the relevant animal models for evaluating the effects of an scFc Type III Interferon fusion protein on such cancers, are discussed in further detail below.

Chronic myeloid leukemia (CML) is a rare type of cancer affecting mostly adults. It is a cancer of granulocytes (one of the main types of white blood cells). In CML many granulocytes are produced and they are released into the blood when they are immature and unable to work properly. The production of other types of blood cells is also disrupted. Normally, white blood cells repair and reproduce themselves in an orderly and controlled manner, but in chronic myeloid leukemia the process gets out of control and the cells continue to divide and mature abnormally. The disease usually develops very slowly, which is why it is called ‘chronic’ myeloid leukemia. Because CML develops (progresses) slowly, it is difficult to detect in its early stages. The symptoms of CML are often vague and non-specific and are caused by the increased number of abnormal white blood cells in the bone marrow and the reduced number of normal blood cells: a feeling of fullness or a tender lump on the left side of the abdomen because of enlargement of the spleen. The effects of an scFc Type III Interferon fusion protein for the treatment of chronic myeloid leukemia can be evaluated in a murine chronic myeloid leukemia model similar to that described in Ren, R., Oncogene. 2002 Dec. 9; 21 (56):8629-42; Wertheim et al., Oncogene. 2002 Dec. 9; 21 (56):8612-28; and Wolff et al., Blood. 2001 Nov. 1; 98 (9):2808-16.

Multiple myeloma is a type of cancer that affects the plasma cells by causing their unregulated production. Myeloma cells tend to collect in the bone marrow and in the hard, outer part of bones. Myeloma cells can form a single mass, or tumor called a plasmacytoma or form many tumors, thus the disease is called multiple myeloma. Those suffering from multiple myeloma have an abnormally large number of identical plasma cells, and also have too much of one type of antibody. These myeloma cells and antibodies can cause a number of serious medical problems: (1) myeloma cells damage and weaken bones, causing pain and sometimes fractures; (2) hypocalcaemia, which often results in loss of appetite, nausea, thirst, fatigue, muscle weakness, restlessness, and confusion; (3) myeloma cells prevent the bone marrow from forming normal plasma cells and other white blood cells that are important to the immune system; (4) myeloma cells prevent the growth of new red blood cells, causing anemia; and (5) kidney problems. Symptoms of multiple myeloma depend on how advanced is the disease. In the earliest stage of the disease a patient may be asymptomatic. Symptoms include bone pain, broken bones, weakness, fatigue, weight loss, repeated infections, nausea, vomiting, constipation, problems with urination, and weakness or numbness in the legs. The effects of an scFc Type III Interferon fusion protein designed to treat multiple myeloma can be evaluated in a multiple myeloma murine model similar to that described in Oyajobi et al., Blood. 2003 Jul. 1; 102 (1):311-9; Croucher et al., J Bone Miner Res. 2003 March; 18 (3):482-92; Asosingh et al., Hematol J. 2000; 1 (5):351-6; and Miyakawa et al., Biochem Biophys Res Commun. 2004 Jan. 9; 313 (2):258-62.

Lymphomas are a type of cancer of the lymphatic system. There are two main types of lymphoma. One is called Hodgkin's disease (named after Dr Hodgkin, who first described it). The other is called non-Hodgkin's lymphoma. There are about 20 different types of non-Hodgkin's lymphoma. In most cases of Hodgkin's disease, a particular cell known as the Reed-Sternberg cell is found in the biopsies. This cell is not usually found in other lymphomas, so they are called non-Hodgkin's lymphoma. Symptoms of a non-Hodgkin's lymphoma is a painless swelling of a lymph node in the neck, armpit or groin; night sweats or unexplained high temperatures (fever); loss of appetite, unexplained weight loss and excessive tiredness. The effects of an scFc Type III Interferon fusion protein designed to treat a lymphoma, particularly a non-Hodgkin's lymphoma, can be evaluated in a murine non-Hodgkin's lymphoma model similar to that described in Ansell et al., Leukemia. 2004 March; 18 (3):616-23; De Jonge et al., J Immunol. 1998 Aug. 1; 161 (3):1454-61; and Slavin et al., Nature. 1978 Apr. 13; 272 (5654):624-6.

The classification of Non-Hodgkin's lymphomas most commonly used is the REAL classification system (Ottensmeier, Chemico-Biological Interactions 135-136:653-664, 2001.) Specific immunological markers have been identified for classifications of lymphomas. For example, follicular lymphoma markers include CD20+, CD3−, CD10+, CD5−; small lymphocytic lymphoma markers include CD20+, CD3−, CD10−, CD5+, CD23+; marginal zone B cell lymphoma markers include CD20+, CD3−, CD10−, CD23−; diffuse large B cell lymphoma markers include CD20+, CD3−; mantle cell lymphoma markers include CD20+, CD3−, CD10−, CD5+, CD23+; peripheral T-cell lymphoma markers include CD20−, CD3+; primary mediastinal large B cell lymphoma markers include CD20+, CD3−, lymphoblastic lymphoma markers include CD20−, CD3+, Tdt+, and Burkitt's lymphoma markers include CD20+, CD3−, CD10+, CD5− (Decision Resourses, Non-Hodgkins Lymphoma, Waltham, Mass., February 2002).

Melanomas: Superficial spreading melanoma is the most common type of melanoma. About 7 out of 10 (70%) are this type. The most common place in women is on the legs, while in men it is more common on the trunk, particularly the back. They tend to start by spreading out across the surface of the skin: this is known as the radial growth phase. The melanoma will then start to grow down deeper into the layers of the skin, and eventually into the bloodstream or lymph system to other parts of the body. Nodular melanoma occurs most often on the chest or back. It tends to grow deeper into the skin quite quickly if it is not removed. This type of melanoma is often raised above the rest of the skin surface and feels like a bump. It may be very dark brown-black or black. Lentigo maligna melanoma is most commonly found on the face. It grows slowly and may take several years to develop. Acral melanoma is usually found on the palms of the hands, soles of the feet or around the toenails. Other very rare types of melanoma of the skin include amelanotic melanoma (in which the melanoma loses its pigment and appears as a white area) and desmoplastic melanoma (which contains fibrous scar tissue). Malignant melanoma can start in parts of the body other than the skin but this is very rare. The parts of the body that may be affected are the eye, the mouth, under the fingernails (known as subungual melanoma) the vulval or vaginal tissues, or internally. The effects of an scFc Type III Interferon fusion protein designed to treat melanoma can be evaluated in a murine melanoma model similar to that described in Hermans et al., Cancer Res. 2003 Dec. 1; 63 (23):8408-13; Ramont et al., Exp Cell Res. 2003 Nov. 15; 291 (1):1-10; Safwat et al., J Exp Ther Oncol. 2003 July-August ; 3 (4):161-8; and Fidler, I. J., Nat New Biol. 1973 April 4; 242 (118):148-9.

Renal cell carcinoma, a form of kidney cancer that involves cancerous changes in the cells of the renal tubule. The first symptom is usually blood in the urine. The cancer metastasizes or spreads easily; most often spreading to the lungs and other organs. The effects of an scFc Type III Interferon fusion protein designed to treat melanoma can be evaluated in a murine renal cell carcinoma model similar to that described in Sayers et al., Cancer Res. 1990 Sep. 1; 50 (17):5414-20; Salup et al., Immunol. 1987 Jan. 15; 138 (2):641-7; and Luan et al., Transplantation. 2002 May 27; 73 (10):1565-72.

Cervical cancer, also called cervical carcinoma, develops from abnormal cells on the surface of the cervix. Cervical cancer is usually preceded by dysplasia, precancerous changes in the cells on the surface of the cervix. These abnormal cells can progress to invasive cancer. Once the cancer appears it can progress through four stages. The stages are defined by the extent of spread of the cancer. There are two main types of cervical cancer: (1) squamous type (epidermoid cancer), which may be diagnosed at an early stage by a pap smear; and (2) adenocarcinoma, which is usually detected by a pap smear and pelvic exam. Later stages of cervical cancer cause abnormal vaginal bleeding or a bloodstained discharge at unexpected times, such as between menstrual periods, after intercourse, or after menopause. Abnormal vaginal discharge may be cloudy or bloody or may contain mucus with a bad odor. Advanced stages of the cancer may cause pain. The effects of an scFc Type III Interferon fusion protein designed to treat cervical cancer can be evaluated in a murine cervical cancer model similar to that described in Ahn et al., Hum Gene Ther. 2003 Oct. 10; 14 (15):1389-99; Hussain et al., Oncology. 1992; 49 (3):237-40; and Sengupta et al., Oncology. 1991; 48 (3):258-61.

Head and Neck tumors: Most cancers of the head and neck are of a type called carcinoma (in particular squamous cell carcinoma). Carcinomas of the head and neck start in the cells that form the lining of the mouth, nose, throat or ear, or the surface layer covering the tongue. However, cancers of the head and neck can develop from other types of cells. Lymphoma develops from the cells of the lymphatic system. Sarcoma develops from the supportive cells which make up muscles, cartilage or blood vessels. Melanoma starts from cells called melanocytes, which give colour to the eyes and skin. The symptoms of a head and neck cancer will depend on its location—for example, cancer of the tongue may cause some slurring of speech. The most common symptoms are an ulcer or sore area in the head or neck that does not heal within a few weeks; difficulty in swallowing, or pain when chewing or swallowing; trouble with breathing or speaking, such as persistent noisy breathing, slurred speech or a hoarse voice; a numb feeling in the mouth; a persistent blocked nose, or nose bleeds; persistent earache, ringing in the ear, or difficulty in hearing; a swelling or lump in the mouth or neck; pain in the face or upper jaw; in people who smoke or chew tobacco, pre-cancerous changes can occur in the lining of the mouth, or on the tongue. These can appear as persistent white patches (leukoplakia) or red patches (erythroplakia). They are usually painless but can sometimes be sore and may bleed (Cancerbacup Internet website). The effects of an scFc Type III Interferon fusion protein designed for treating head and neck cancers can be evaluated in a murine head and neck tumor model similar to that described in Kuriakose et al., Head Neck. 2000 January; 22 (1):57-63; Cao et al., Clin Cancer Res. 1999 July; 5 (7):1925-34; Hier et al., Laryngoscope. 1995 October; 105 (10):1077-80; Braakhuis et al., Cancer Res. 1991 Jan. 1; 51 (1):211-4; Baker, S. R., Laryngoscope. 1985 January; 95 (1):43-56; and Dong et al., Cancer Gene Ther. 2003 February; 10 (2):96-104.

Brain Cancer: Tumors that begin in brain tissue are known as primary tumors of the brain. Primary brain tumors are named according to the type of cells or the part of the brain in which they begin. The most common primary brain tumors are gliomas. They begin in glial cells. There are many types of gliomas. Astrocytomas arise from star-shaped glial cells called astrocytes. In adults, astrocytomas most often arise in the cerebrum. In children, they occur in the brain stem, the cerebrum, and the cerebellum. A grade III astrocytoma is sometimes called an anaplastic astrocytoma. A grade IV astrocytoma is usually called a glioblastoma multiforme. Brain stem gliomas occur in the lowest part of the brain. Ependymomas arise from cells that line the ventricles or the central canal of the spinal cord. Oligodendrogliomas arise from cells that make the fatty substance that covers and protects nerves. These tumors usually occur in the cerebrum. They grow slowly and usually do not spread into surrounding brain tissue. The symptoms of brain tumors depend on tumor size, type, and location. Symptoms may be caused when a tumor presses on a nerve or damages a certain area of the brain. They also may be caused when the brain swells or fluid builds up within the skull. These are the most common symptoms of brain tumors: Headaches; Nausea or vomiting; Changes in speech, vision, or hearing; Problems balancing or walking; Changes in mood, personality, or ability to concentrate; Problems with memory; Muscle jerking or twitching (seizures or convulsions); and Numbness or tingling in the arms or legs. The effects of an scFc Type III Interferon fusion protein designed to treat brain cancer can be evaluated in a glioma animal model similar to that described in Schueneman et al., Cancer Res. 2003 Jul. 15; 63 (14):4009-16; Martinet et al., Eur J Surg Oncol. 2003 May; 29 (4):351-7; Bello et al., Clin Cancer Res. 2002 November; 8 (11):3539-48; Ishikawa et al., Cancer Sci. 2004 January; 95 (1):98-103; Degen et al., J Neurosurg. 2003 November; 99 (5):893-8; Engelhard et al., Neurosurgery. 2001 March; 48 (3):616-24; Watanabe et al., Neural Res. 2002 July; 24 (5):485-90; and Lumniczky et al., Cancer Gene Ther. 2002 January; 9 (1):44-52.

Thyroid Cancer: Papillary and follicular thyroid cancers account for 80 to 90 percent of all thyroid cancers. Both types begin in the follicular cells of the thyroid. Most papillary and follicular thyroid cancers tend to grow slowly. Medullary thyroid cancer accounts for 5 to 10 percent of thyroid cancer cases. Anaplastic thyroid cancer is the least common type of thyroid cancer (only 1 to 2 percent of cases). The cancer cells are highly abnormal and difficult to recognize. This type of cancer is usually very hard to control because the cancer cells tend to grow and spread very quickly. Early thyroid cancer often does not cause symptoms. But as the cancer grows, symptoms may include: A lump, or nodule, in the front of the neck near the prominentia laryngea; Hoarseness or difficulty speaking in a normal voice; Swollen lymph nodes, especially in the neck; Difficulty swallowing or breathing; or Pain in the throat or neck. The effects of an scFc Type III Interferon fusion protein designed for the treatment of thyroid cancer can be evaluated in a murine or rat thyroid tumor model similar to that described in Quidville et al., Endocrinology. 2004 May; 145 (5):2561-71 (mouse model); Cranston et al., Cancer Res. 2003 Aug. 15; 63 (16):4777-80 (mouse model); Zhang et al., Clin Endocrinol (Oxf). 2000 June; 52 (6):687-94 (rat model); and Zhang et al., Endocrinology. 1999 May; 140 (5):2152-8 (rat model).

Liver Cancer: There are two different types of primary liver cancer. The most common kind is called hepatoma or hepatocellular carcinoma (HCC), and arises from the main cells of the liver (the hepatocytes). This type is usually confined to the liver, although occasionally it spreads to other organs. There is also a rarer sub-type of hepatoma called Fibrolamellar hepatoma. The other type of primary liver cancer is called cholangiocarcinoma or bile duct cancer, because it starts in the cells lining the bile ducts. Most people who develop hepatoma usually also have a condition called cirrhosis of the liver. Infection with either the hepatitis B or hepatitis C virus can lead to liver cancer, and can also be the cause of cirrhosis, which increases the risk of developing hepatoma. People who have a rare condition called haemochromatosis, which causes excess deposits of iron in the body, have a higher chance of developing hepatoma. Thus, an scFc molecule of the present invention may be used to treat, prevent, inhibit the progression of, delay the onset of, and/or reduce the severity or inhibit at least one of the conditions or symptoms associated with hepatocellular carcinoma. The effects of an scFc Type III Interferon fusion protein designed to treat liver cancer can be evaluated in a hepatocellular carcinoma transgenic mouse model, which includes the overexpression of transforming growth factor-.alpha. (TFG-.alpha.) alone (Jhappan et al., Cell, 61:1137-1146 (1990); Sandgren et al., Mol. Cell Biol., 13:320-330 (1993); Sandgren et al., Oncogene, 4:715-724 (1989); and Lee et al., Cancer Res., 52:5162:5170 (1992)) or in combination with c-myc (Murakami et al., Cancer Res., 53:1719-1723 (1993), mutated H-ras (Saitoh et al., Oncogene, 5:1195-2000 (1990)), hepatitis B viral genes encoding HbsAg and HBx (Toshkov et al., Hepatology, 20:1162-1172 (1994) and Koike et al., Hepatology, 19:810-819 (1994)), SV40 large T antigen (Sepulveda et al., Cancer Res., 49:6108-6117 (1989) and Schirmacher et al., Am. J. Pathol., 139:231-241 (1991)) and FGF19 (Nicholes et al., American Journal of Pathology, 160 (6):2295-2307 (June 2002)).

Lung cancer: The effects of an scFc molecule designed to treat a lung cancer can be evaluated in a human small/non-small cell lung carcinoma xenograft model. Briefly, human tumors are grafted into immunodecicient mice and these mice are treated with an scFc Type III Interferon fusion protein alone or in combination with other agents which can be used to demonstrate the efficacy of the treatment by evaluating tumor growth (Nemati et al., Clin Cancer Res. 2000 May; 6 (5):2075-86; and Hu et al., Clin Cancer Res. 2004 Nov. 15; 10 (22):7662-70).

### 2. Endpoints And Anti-tumor Activity For Solid Tumors

While each protocol may define tumor response assessments differently, the RECIST (Response evaluation Criteria in solid tumors) criteria is currently considered to be the recommended guidelines for assessment of tumor response by the National Cancer Institute (see Therasse et al., J. Natl. Cancer Inst. 92:205-216, 2000). According to the RECIST criteria tumor response means a reduction or elimination of all measurable lesions or metastases. Disease is generally considered measurable if it comprises lesions that can be accurately measured in atleast one dimension as >20 mm with conventional techniques or >10 mm with spiral CT scan with clearly defined margins by medical photograph or X-ray, computerized axial tomography (CT), magnetic resonance imaging (MRI), or clinical examination (if lesions are superficial). Non-measurable disease means the disease comprises of lesions <20 mm with conventional techniques or <10 mm with spiral CT scan, and truely non-measurable lesions (too small to accurately measure). Non-measureable disease includes pleural effusions, ascites, and disease documented by indirect evidence.

The criteria for objective status are required for protocols to assess solid tumor response. Representative criteria include the following: (1) Complete Response (CR) defined as complete disappearance of all measurable and evaluable disease. No new lesions. No disease related symptoms. No evidence of non-evaluable disease; (2) Partial Response (PR) defined as greater than or equal to 50% decrease from baseline in the sum of products of perpendicular diameters of all measurable lesions. No progression of evaluable disease. No new lesions. Applies to patients with at least one measurable lesion; (3) Progression defined as 50% or an increase of 10 cm.sup.2 in the sum of products of measurable lesions over the smallest sum observed using same techniques as baseline, or clear worsening of any evaluable disease, or reappearance of any lesion which had disappeared, or appearance of any new lesion, or failure to return for evaluation due to death or deteriorating condition (unless unrelated to this cancer); (4) Stable or No Response defined as not qualifying for CR, PR, or Progression. (See, Clinical Research Associates Manual, ibid.)

Additional endpoints that are accepted within the oncology art include overall survival (OS), disease-free survival (DFS), objective response rate (ORR), time to progression (TTP), and progression-free survival (PFS) (see, Guidance for Industry: Clinical Trial Endpoints for the Approval of Cancer Drugs and Biologics, April 2005, Center for Drug Evaluation and Research, FDA, Rockville, Md.)

a. Chemotherapy Combinations

In certain embodiments, an scFc Type III Interferon fusion protein is administered in combination with one or more chemotherapeutic agents. Chemotherapeutic agents have different modes of actions, for example, by influencing either DNA or RNA and interfering with cell cycle replication. Examples of chemotherapeutic agents that act at the DNA level or on the RNA level are anti-metabolites (such as Azathioprine, Cytarabine, Fludarabine phosphate, Fludarabine, Gemcitabine, cytarabine, Cladribine, capecitabine 6-mercaptopurine, 6-thioguanine, methotrexate, 5-fluoroouracil and hyroxyurea); alkylating agents (such as Melphalan, Busulfan, Cis-platin, Carboplatin, Cyclophosphamide, Ifosphamide, Dacarabazine, Procarbazine, Chlorambucil, Thiotepa, Lomustine, Temozolamide); anti-mitotic agents (such as Vinorelbine, Vincristine, Vinblastine, Docetaxel, Paclitaxel); topoisomerase inhibitors (such as Doxorubincin, Amsacrine, Irinotecan, Daunorubicin, Epirubicin, Mitomycin, Mitoxantrone, Idarubicin, Teniposide, Etoposide, Topotecan); antibiotics (such as actinomycin and bleomycin); asparaginase; anthracyclines or taxanes.

b. Radiotherapy Combinations

In some variations, an scFc Type III Interferon fusion protein is administered in combination with radiotherapy. Certain tumors can be treated with radiation or radiopharmaceuticals. Radiation therapy is generally used to treat unresectable or inoperable tumors and/or tumor metastases. Radiotherapy is typically delivered in three ways. External beam irradiation is administered at distance from the body and includes gamma rays (60 Co) and X-rays. Brachytherapy uses sources, for example .sup.60Co, .sup.137Cs, .sup.192Ir, or .sup.125I, with or in contact with a target tissue.

c. Hormonal Agent Combinations

In some embodiments, an scFc Type III Interferon fusion protein is administered in combination with a hormone or anti-hormone. Certain cancers are associated with hormonal dependency and include, for example, ovarian cancer, breast cancer, and prostate cancer. Hormonal-dependent cancer treatment may comprise use of anti-androgen or anti-estrogen compounds. Hormones and anti-hormones used in cancer therapy include Estramustine phosphate, Polyestradiol phosphate, Estradiol, Anastrozole, Exemestane, Letrozole, Tamoxifen, Megestrol acetate, Medroxyprogesterone acetate, Octreotide, Cyproterone acetate, Bicaltumide, Flutamide, Tritorelin, Leuprorelin, Buserelin and Goserelin.

C. Inflammation And Autoimmunity Treatment

Diseases of the immune system are significant healthcare problems that are growing at epidemic proportions. As such, they require novel, aggressive approaches to the development of new therapeutic agents. Standard therapy for autoimmune disease has been high dose, long-term systemic corticosteroids and immunosuppressive agents. The drugs used fall into three major categories: (1) glucocorticoids, such as prednisone and prednisolone; (2) calcineurin inhibitors, such as cyclosporine and tacrolimus; and (3) antiproliferative/antimetabolic agents such as azathioprine, sirolimus, and mycophenolate mofetil. Although these drugs have met with high clinical success in treating a number of autoimmune conditions, such therapies require lifelong use and act nonspecifically to suppress the entire immune system. The patients are thus exposed to significantly higher risks of infection and cancer. The calcineurin inhibitors and steroids are also nephrotoxic and diabetogenic, which has limited their clinical utility.

In addition to the conventional therapies for autoimmune disease, monoclonal antibodies and soluble receptors that target cytokines and their receptors have shown efficacy in a variety of autoimmune and inflammation diseases such as rheumatoid arthritis, organ transplantation, and Crohn's disease. Some of the agents include infliximab (REMICADE) and etanercept (ENBREL) that target tumor necrosis factor (TNF), muromonab-CD3 (ORTHOCLONE OKT3) that targets the T cell antigen CD3, and daclizumab (ZENAPAX) that binds to CD25 on activated T cells, inhibiting signaling through this pathway. While efficacious in treating certain inflammatory conditions, use of these drugs has been limited by side effects including the “cytokine release syndrome” and an increased risk of infection.

Passive immunization with intravenous immunoglobulin (WIG) was licensed in the United States in 1981 for replacement therapy in patients with primary antibody deficiencies. IVIG is obtained from the plasma of large numbers (10,000-20,000) of healthy donors by cold ethanol fractionation. Commonly used IVIG preparations include Sandoglobulin, Flebogamma, Gammagard, Octagam, and Vigam S.

Subsequent investigation showed that WIG was also effective in ameliorating autoimmune symptoms in Kawasaki's disease and immune thrombocytopenia purpura. WIG has also been shown to reduce inflammation in adult dermatomyositis, Guillian-Barre syndrome, chronic inflammatory demyelinating polyneuropathies, multiple sclerosis, vasculitis, uveitis, myasthenia gravis, and in the Lambert-Eaton syndrome. Numerous mechanisms have been proposed to explain the mode of action of IVIG, including regulation of Fc gamma receptor expression, increased clearance of pathogenic antibodies due to saturation of the neonatal Fc receptor FcRn, attenuation of complement-mediated damage, and modulation of T and B cells or the reticuloendothelial system. Since Fc domains purified from IVIG are as active as intact IgG in a number of in vitro and in vivo models of inflammation, it is well accepted that the anti-inflammatory properties of WIG reside in the Fc domain of the IgG. In general, efficacy is seen when only large amounts of WIG are infused into a patient, with an average dose of 2 g/kg/month used in autoimmune disease.

The common (1-10% of patients) side effects of IVIG treatment include flushing, fever, myalgia, back pain, headache, nausea, vomiting, arthralgia, and dizziness. Uncommon (0.1-1% of patients) side effects include anaphylaxis, aseptic meningitis, acute renal failure, haemolytic anemia, and eczema. Although WIG is generally considered safe, the pooled human plasma source is considered to be risk factor for transfer of infectious agents. Thus, the use of IVIG is limited by its availability, high cost ($100/gm, including infusion cost), and the potential for severe adverse reactions. Thus, it would be significantly advantageous to develop a therapeutic that offered the efficacy of IVIG without the numerous issues described above (undue side effects and cost/availability issues).

As such, the present invention concerns compositions and methods useful for the diagnosis and treatment of immune related disease in mammals, including humans. The present invention is based on the identification of scFc Type III Interferon fusion proteins which inhibit the immune response in mammals and may be used to treat inflammatory and immune diseases or conditions such as acute or chronic inflammation, ulcerative colitis, chronic bronchitis, asthma, emphysema, myositis, polymyositis, immune dysregulation diseases, cachexia, septicemia, atherosclerosis, psoriasis, psoriatic arthritis, atopic dermatitis, inflammatory skin conditions, rheumatoid arthritis, inflammatory bowel disease (IBD), Crohn's Disease, diverticulosis, pancreatitis, type I diabetes (IDDM), pancreatic cancer, pancreatitis, Graves Disease, colon and intestinal cancer, autoimmune disease, sepsis, organ or bone marrow transplant rejection; inflammation due to endotoxemia, trauma, surgery or infection; amyloidosis; splenomegaly; graft versus host disease; and where inhibition of inflammation, immune suppression, reduction of proliferation of hematopoietic, immune, inflammatory or lymphoid cells, macrophages, T-cells (including Th1 and Th2 cells), suppression of immune response to a pathogen or antigen. Immunotherapy of autoimmune disorders using antibodies which target B-cells is described in PCT Application Publication No. WO 00174718. Exemplary autoimmune diseases are acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcalnephritis, erythema nodosurn, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitisubiterans, Sjogren's syndrome, primary biliary cirrhosis, Hashimoto's thyroiditis, thyrotoxicosis, scleroderma, chronic active hepatitis, polymyositis/dermatomyositis, polychondritis, parnphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis, and fibrosing alveolitis.

Inflammation is a protective response by an organism to fend off an invading agent. Inflammation is a cascading event that involves many cellular and humoral mediators. On one hand, suppression of inflammatory responses can leave a host immunocompromised; however, if left unchecked, inflammation can lead to serious complications including chronic inflammatory diseases (e.g., psoriasis, arthritis, rheumatoid arthritis, multiple sclerosis, inflammatory bowel disease and the like), septic shock and multiple organ failure. Importantly, these diverse disease states share common inflammatory mediators. The collective diseases that are characterized by inflammation have a large impact on human morbidity and mortality. Therefore it is clear that the scFc Type III Interferon fusion proteins of the present invention could have crucial therapeutic potential for a vast number of human and animal diseases, from asthma and allergy to autoimmunity and septic shock.

There are several in vivo models for testing multiple sclerosis that are known to those skilled in the art. Experimental allergic encephalomyelitis (EAE) is a mouse model for human multiple sclerosis (MS) (See, for example, Gold et al., Mol. Med. Today, 6:88-91, 2000; Anderton et al., Immunol. Rev., 169:123-137, 1999; and US Patent Publication No. 2007-0020227).

1. Arthritis

Arthritis, including osteoarthritis, rheumatoid arthritis, arthritic joints as a result of injury, and the like, are common inflammatory conditions which would benefit from the therapeutic use of the scFc Type III Interferons fusion proteins of the present invention. For example, rheumatoid arthritis (RA) is a systemic disease that affects the entire body and is one of the most common forms of arthritis. It is characterized by the inflammation of the membrane lining the joint, which causes pain, stiffness, warmth, redness and swelling. Inflammatory cells release enzymes that may digest bone and cartilage. As a result of rheumatoid arthritis, the inflamed joint lining, the synovium, can invade and damage bone and cartilage leading to joint deterioration and severe pain amongst other physiologic effects. The involved joint can lose its shape and alignment, resulting in pain and loss of movement.

Rheumatoid arthritis (RA) is an immune-mediated disease particularly characterized by inflammation and subsequent tissue damage leading to severe disability and increased mortality. A variety of cytokines are produced locally in the rheumatoid joints. Numerous studies have demonstrated that IL-1 and TNF-alpha, two prototypic pro-inflammatory cytokines, play an important role in the mechanisms involved in synovial inflammation and in progressive joint destruction. Indeed, the administration of TNF-alpha and IL-1 inhibitors in patients with RA has led to a dramatic improvement of clinical and biological signs of inflammation and a reduction of radiological signs of bone erosion and cartilage destruction. However, despite these encouraging results, a significant percentage of patients do not respond to these agents, suggesting that other mediators are also involved in the pathophysiology of arthritis (Gabay, Expert. Opin. Biol. Ther. 2 (2):135-149, 2002).

There are several animal models for rheumatoid arthritis known in the art. For example, in the collagen-induced arthritis (CIA) model, mice develop chronic inflammatory arthritis that closely resembles human rheumatoid arthritis. Since CIA shares similar immunological and pathological features with RA, this makes it an ideal model for screening potential human anti-inflammatory compounds. The CIA model is a well-known model in mice that depends on both an immune response, and an inflammatory response, in order to occur. The immune response comprises the interaction of B-cells and CD4+ T-cells in response to collagen, which is given as antigen, and leads to the production of anti-collagen antibodies. The inflammatory phase is the result of tissue responses from mediators of inflammation, as a consequence of some of these antibodies cross-reacting to the mouse's native collagen and activating the complement cascade. An advantage in using the CIA model is that the basic mechanisms of pathogenesis are known. The relevant T-cell and B-cell epitopes on type II collagen have been identified, and various immunological (e.g., delayed-type hypersensitivity and anti-collagen antibody) and inflammatory (e.g., cytokines, chemokines, and matrix-degrading enzymes) parameters relating to immune-mediated arthritis have been determined, and can thus be used to assess test compound efficacy in the CIA model (Wooley, Curr. Opin. Rheum. 3:407-20, 1999; Williams et al., Immunol. 89:9784-788, 1992; Myers et al., Life Sci. 61:1861-78, 1997; and Wang et al., Immunol. 92:8955-959, 1995).

The administration of the scFc Type III Interferon fusion proteins of the invention to these CIA model mice is used to evaluate the use of such molecules as a therapeutic useful in ameliorating symptoms and altering the course of disease. By way of example and without limitation, the injection of 10-200 .micro.g of such an antibody fragment of the present invention per mouse (one to seven times a week for up to but not limited to 4 weeks via s.c., i.p., or i.m route of administration) can significantly reduce the disease score (paw score, incidence of inflammation, or disease). Depending on the initiation of administration (e.g. prior to or at the time of collagen immunization, or at any time point following the second collagen immunization, including those time points at which the disease has already progressed), such antibody fragments can be efficacious in preventing rheumatoid arthritis, as well as preventing its progression.

2. Endotoxemia

Endotoxemia is a severe condition commonly resulting from infectious agents such as bacteria and other infectious disease agents, sepsis, toxic shock syndrome, or in immunocompromised patients subjected to opportunistic infections, and the like. Therapeutically useful of anti-inflammatory proteins, such as antibodies of the invention, could aid in preventing and treating endotoxemia in humans and animals. Such scFc Type III Interferon fusion proteins could serve as a valuable therapeutic to reduce inflammation and pathological effects in endotoxemia.

Lipopolysaccharide (LPS) induced endotoxemia engages many of the proinflammatory mediators that produce pathological effects in the infectious diseases and LPS induced endotoxemia in rodents is a widely used and acceptable model for studying the pharmacological effects of potential pro-inflammatory or immunomodulating agents. LPS, produced in gram-negative bacteria, is a major causative agent in the pathogenesis of septic shock (Glausner et al., Lancet 338:732, 1991). A shock-like state can indeed be induced experimentally by a single injection of LPS into animals. Molecules produced by cells responding to LPS can target pathogens directly or indirectly. Although these biological responses protect the host against invading pathogens, they may also cause harm. Thus, massive stimulation of innate immunity, occurring as a result of severe Gram-negative bacterial infection, leads to excess production of cytokines and other molecules, and the development of a fatal syndrome, septic shock syndrome, which is characterized by fever, hypotension, disseminated intravascular coagulation, and multiple organ failure (Dumitru et al. Cell 103:1071-1083, 2000).

These toxic effects of LPS are mostly related to macrophage activation leading to the release of multiple inflammatory mediators. Among these mediators, TNF appears to play a crucial role, as indicated by the prevention of LPS toxicity by the administration of neutralizing anti-TNF antibodies (Beutler et al., Science 229:869, 1985). It is well established that 1 .micro.g injection of E. coli LPS into a C57B1/6 mouse will result in significant increases in circulating IL-6, TNF-alpha, IL-1, and acute phase proteins (for example, SAA) approximately 2 hours post injection. The toxicity of LPS appears to be mediated by these cytokines as passive immunization against these mediators can result in decreased mortality (Beutler et al., Science 229:869, 1985). The potential immunointervention strategies for the prevention and/or treatment of septic shock include anti-TNF mAb, IL-1 receptor antagonist, LIF, IL-10, and G-CSF.

The administration of scFc Type III Interferon fusion proteins of the invention to an LPS-induced model may be used to evaluate the use of such antibody fragments to ameliorate symptoms and alter the course of LPS-induced disease. Moreover, results showing inhibition of immune response by such antibody fragments of the invention provide proof of concept that such scFc Type III Interferon fusion proteins can also be used to ameliorate symptoms in the LPS-induced model and alter the course of disease. The model will show induction of disease specific cytokines by LPS injection and the potential treatment of disease by such antibody fragments. Since LPS induces the production of pro-inflammatory factors possibly contributing to the pathology of endotoxemia, the neutralization of pro-inflammatory factors by scFc Type III Interferon fusion proteins of the invention can be used to reduce the symptoms of endotoxemia, such as seen in endotoxic shock.

3. Inflammatory Bowel Disease (IBD)

In the United States approximately 500,000 people suffer from Inflammatory Bowel Disease (IBD) which can affect either colon and rectum (Ulcerative colitis) or both, small and large intestine (Crohn's Disease). The pathogenesis of these diseases is unclear, but they involve chronic inflammation of the affected tissues. scFc Type III Interferon fusion proteins of the invention could serve as a valuable therapeutic to reduce inflammation and pathological effects in IBD and related diseases.

Ulcerative colitis (UC) is an inflammatory disease of the large intestine, commonly called the colon, characterized by inflammation and ulceration of the mucosa or innermost lining of the colon. This inflammation causes the colon to empty frequently, resulting in diarrhea. Symptoms include loosening of the stool and associated abdominal cramping, fever and weight loss. Although the exact cause of UC is unknown, recent research suggests that the body's natural defenses are operating against proteins in the body which the body thinks are foreign (an “autoimmune reaction”). Perhaps because they resemble bacterial proteins in the gut, these proteins may either instigate or stimulate the inflammatory process that begins to destroy the lining of the colon. As the lining of the colon is destroyed, ulcers form releasing mucus, pus and blood. The disease usually begins in the rectal area and may eventually extend through the entire large bowel. Repeated episodes of inflammation lead to thickening of the wall of the intestine and rectum with scar tissue. Death of colon tissue or sepsis may occur with severe disease. The symptoms of ulcerative colitis vary in severity and their onset may be gradual or sudden. Attacks may be provoked by many factors, including respiratory infections or stress.

Although there is currently no cure for UC available, treatments are focused on suppressing the abnormal inflammatory process in the colon lining. Treatments including corticosteroids, immunosuppressives (eg. azathioprine, mercaptopurine, and methotrexate) and aminosalicytates are available to treat the disease. However, the long-term use of immunosuppressives such as corticosteroids and azathioprine can result in serious side effects including thinning of bones, cataracts, infection, and liver and bone marrow effects. In the patients in whom current therapies are not successful, surgery is an option. The surgery involves the removal of the entire colon and the rectum.

There are several animal models that can partially mimic chronic ulcerative colitis. The most widely used model is the 2,4,6-trinitrobenesulfonic acid/ethanol (TNBS) induced colitis model, which induces chronic inflammation and ulceration in the colon. When TNBS is introduced into the colon of susceptible mice via intra-rectal instillation, it induces T-cell mediated immune response in the colonic mucosa, in this case leading to a massive mucosal inflammation characterized by the dense infiltration of T-cells and macrophages throughout the entire wall of the large bowel. Moreover, this histopathologic picture is accompanied by the clinical picture of progressive weight loss (wasting), bloody diarrhea, rectal prolapse, and large bowel wall thickening (Neurath et al. Intern. Rev. Immunol. 19:51-62, 2000).

Another colitis model uses dextran sulfate sodium (DSS), which induces an acute colitis manifested by bloody diarrhea, weight loss, shortening of the colon and mucosal ulceration with neutrophil infiltration. DSS-induced colitis is characterized histologically by infiltration of inflammatory cells into the lamina propria, with lymphoid hyperplasia, focal crypt damage, and epithelial ulceration. These changes are thought to develop due to a toxic effect of DSS on the epithelium and by phagocytosis of lamina propria cells and production of TNF-alpha and IFN-gamma. Despite its common use, several issues regarding the mechanisms of DSS about the relevance to the human disease remain unresolved. DSS is regarded as a T cell-independent model because it is observed in T cell-deficient animals such as SCID mice.

The administration of scFc Type III Interferon fusion proteins of the invention to these TNBS or DSS models can be used to evaluate the use such antibody fragments to ameliorate symptoms and alter the course of gastrointestinal disease.

4. Psoriasis

Psoriasis is a chronic skin condition that affects more than seven million Americans. Psoriasis occurs when new skin cells grow abnormally, resulting in inflamed, swollen, and scaly patches of skin where the old skin has not shed quickly enough. Plaque psoriasis, the most common form, is characterized by inflamed patches of skin (“lesions”) topped with silvery white scales. Psoriasis may be limited to a few plaques or involve moderate to extensive areas of skin, appearing most commonly on the scalp, knees, elbows and trunk. Although it is highly visible, psoriasis is not a contagious disease. The pathogenesis of the diseases involves chronic inflammation of the affected tissues. The scFc Type III Interferon fusion proteins of the invention could serve as a valuable therapeutic to reduce inflammation and pathological effects in psoriasis, other inflammatory skin diseases, skin and mucosal allergies, and related diseases.

Psoriasis is a T-cell mediated inflammatory disorder of the skin that can cause considerable discomfort. It is a disease for which there is no cure and affects people of all ages. Psoriasis affects approximately two percent of the populations of European and North America. Although individuals with mild psoriasis can often control their disease with topical agents, more than one million patients worldwide require ultraviolet or systemic immunosuppressive therapy. Unfortunately, the inconvenience and risks of ultraviolet radiation and the toxicities of many therapies limit their long-term use. Moreover, patients usually have recurrence of psoriasis, and in some cases rebound, shortly after stopping immunosuppressive therapy.

In addition to other disease models described herein, the activity of antibody fragments of the invention on inflammatory tissue derived from human psoriatic lesions can be measured in vivo using a severe combined immune deficient (SCID) mouse model. Several mouse models have been developed in which human cells are implanted into immunodeficient mice (collectively referred to as xenograft models); see, for example, Caftan A R, Douglas E, Leuk. Res. 18:513-22, 1994 and Flavell, D J, Hematological Oncology 14:67-82, 1996. As an in vivo xenograft model for psoriasis, human psoriatic skin tissue is implanted into the SCID mouse model, and challenged with an appropriate antagonist. Moreover, other psoriasis animal models in ther art may be used to evaluate the scFc Type III Interferon fusion proteins of the invention, such as human psoriatic skin grafts implanted into AGR129 mouse model, and challenged with an appropriate antagonist (e.g., see, Boyman, O. et al., J. Exp. Med. Online publication #20031482, 2004, incorporated herein by reference). Similarly, tissues or cells derived from human colitis, IBD, arthritis, or other inflammatory lesions can be used in the SCID model to assess the anti-inflammatory properties of the antibody fragments of the invention described herein.

Therapies designed to abolish, retard, or reduce inflammation using antibody fragments of the invention can be tested by administration of such antibodies to SCID mice bearing human inflammatory tissue (e.g., psoriatic lesions and the like), or other models described herein. Efficacy of treatment is measured and statistically evaluated as increased anti-inflammatory effect within the treated population over time using methods well known in the art. Some exemplary methods include, but are not limited to measuring for example, in a psoriasis model, epidermal thickness, the number of inflammatory cells in the upper dermis, and the grades of parakeratosis. Such methods are known in the art and described herein. For example, see Zeigler, M. et al. Lab Invest 81:1253, 2001; Zollner, T. M. et al. J. Clin. Invest. 109:671, 2002; Yamanaka, N. et al. Microbio.l Immunol 45:507, 2001; Raychaudhuri, S. P. et al. Br. J. Dermatol. 144:931, 2001; Boehncke, W. H et al. Arch. Dermatol. Res. 291:104, 1999; Boehncke, W. H et aL J. Invest. Dermatol. 116:596, 2001; Nickoloff, B. J. et al. Am. J. Pathol. 146:580, 1995; Boehncke, W. H et al. J. Cutan. Pathol. 24:1, 1997; Sugai, J., M. et al. J. Dermatol. Sci. 17:85, 1998; and Villadsen L. S. et al. J. Clin. Invest. 112:1571, 2003. Inflammation may also be monitored over time using well-known methods such as flow cytometry (or PCR) to quantitate the number of inflammatory or lesional cells present in a sample, score (weight loss, diarrhea, rectal bleeding, colon length) for IBD, paw disease score and inflammation score for CIA RA model.

Moreover, psoriasis is a chronic inflammatory skin disease that is associated with hyperplastic epideiiiial keratinocytes and infiltrating mononuclear cells, including CD430 memory T cells, neutrophils and macrophages (Christophers, Int. Arch. Allergy Immunol., 110:199, 1996). It is currently believed that environmental antigens play a significant role in initiating and contributing to the pathology of the disease. However, it is the loss of tolerance to self-antigens that is thought to mediate the pathology of psoriasis. Dendritic cells and CD4+ T cells are thought to play an important role in antigen presentation and recognition that mediate the immune response leading to the pathology. We have recently developed a model of psoriasis based on the CD4+CD45RB transfer model (Davenport et al., Internat. Immunopharmacol., 2:653-672). The scFc Type III Interferon fusion proteins of the present invention are administered to the mice. Inhibition of disease scores (skin lesions, inflammatory cytokines) indicates the effectiveness of such antibodies in psoriasis.

5. Atopic Dermatitis

AD is a common chronic inflammatory disease that is characterized by hyperactivated cytokines of the helper T cell subset 2 (Th2). Although the exact etiology of AD is unknown, multiple factors have been implicated, including hyperactive Th2 immune responses, autoimmunity, infection, allergens, and genetic predisposition. Key features of the disease include xerosis (dryness of the skin), pruritus (itchiness of the skin), conjunctivitis, inflammatory skin lesions, Staphylococcus aureus infection, elevated blood eosinophilia, elevation of serum IgE and IgG1, and chronic dermatitis with T cell, mast cell, macrophage and eosinophil infiltration. Colonization or infection with S. aureus has been recognized to exacerbate AD and perpetuate chronicity of this skin disease.

AD is often found in patients with asthma and allergic rhinitis, and is frequently the initial manifestation of allergic disease. About 20% of the population in Western Countries suffers from these allergic diseases, and the incidence of AD in developed countries is rising for unknown reasons. AD typically begins in childhood and can often persist through adolescence into adulthood. Current treatments for AD include topical corticosteroids, oral cyclosporin A, non-corticosteroid immunosuppressants such as tacrolimus (FK506 in ointment form), and interferon-gamma. Despite the variety of treatments for AD, many patients' symptoms do not improve, or they have adverse reactions to medications, requiring the search for other, more effective therapeutic agents.

Pharmaceutical Compositons. For pharmaceutical use, scFc Type III Interferon fusion protein is formulated as a pharmaceutical composition. A pharmaceutical composition comprising an scFc Type III Interferon fusion protein can be formulated according to known methods for preparing pharmaceutically useful compositions, whereby the therapeutic molecule is combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. In one embodiment, the scFc Type III Interferon fusion proteins of the present invention are formulated for parenteral, particularly intravenous or subcutaneous, delivery according to conventional methods. Intravenous administration will be by bolus injection, controlled release, e.g, using mini-pumps or other appropriate technology, or by infusion over a typical period of one to several hours. In general, pharmaceutical formulations will include an scFc Type III Interferon fusion protein in combination with a pharmaceutically acceptable carrier, such as saline, buffered saline, 5% dextrose in water or the like. Formulations may further include one or more excipients, preservatives, solubilizers, buffering agents, albumin to prevent protein loss on vial surfaces, etc. When utilizing such a combination therapy, the scFc Type III Interferon fusion proteins may be combined in a single formulation or may be administered in separate formulations. Methods of formulation are well known in the art and are disclosed, for example, in Remington's Pharmaceutical Sciences, Gennaro, ed., Mack Publishing Co., Easton Pa., 1990, which is incorporated herein by reference. Therapeutic doses will generally be in the range of 0.1 to 100 mg/kg of patient weight per day, preferably 0.5-20 mg/kg per day, with the exact dose determined by the clinician according to accepted standards, taking into account the nature and severity of the condition to be treated, patient traits, etc. Determination of dose is within the level of ordinary skill in the art. Monospecific antagonists can be individually formulated or provided in a combined formulation. The scFc Type III Interferon fusion proteins of the present invention can also be administered in combination with other cytokines such as IL-3, -6 and -11; stem cell factor; erythropoietin; G-CSF and GM-CSF.

A composition comprising a scFc Type III Interferon fusion protein is administered to a patient in an effective amount. Generally, the dosage of administered scFc Type III Interferons fusion proteins of the invention will vary depending upon such factors as the patient's age, weight, height, sex, general medical condition and previous medical history. Typically, it is desirable to provide the recipient with a dosage which is in the range of from about 1 pg/kg to 10 mg/kg (amount of agent/body weight of patient), although a lower or higher dosage also may be administered as circumstances dictate.

Administration of the scFc Type III Interferon fusion protein of the invention to a patient can be intravenous, intraarterial, intraperitoneal, intramuscular, subcutaneous, intrapleural, intrathecal, by perfusion through a regional catheter, or by direct intralesional injection. For prevention and treatment purposes, an antagonist may be administered to a patient in a single bolus delivery, via continuous delivery (e.g., continuous transdermal delivery) over an extended time period, or in a repeated administration protocol (e.g., on an hourly, daily, or weekly basis). When administering therapeutic proteins by injection, the administration may be by continuous infusion or by single or multiple boluses. For pharmaceutical use for treatment of neovascular ocular disorders, the scFc molecules are typically formulated for intravitreal injection according to conventional methods.

Additional routes of administration include oral, mucosal-membrane, pulmonary, and transcutaneous. Oral delivery is suitable for polyester microspheres, zein microspheres, proteinoid microspheres, polycyanoacrylate microspheres, and lipid-based systems (see, for example, DiBase and Monet, “Oral Delivery of Microencapsulated Proteins,” in Protein Delivery: Physical Systems, Sanders and Hendren (eds.), pages 255-288 (Plenum Press 1997)). The feasibility of an intranasal delivery is exemplified by such a mode of insulin administration (see, for example, Hinchcliffe and Illum, Adv. Drug Deliv. Rev. 35:199 (1999)). Dry or liquid particles comprising scFc Type III Interferons fusion proteins of the invention can be prepared and inhaled with the aid of dry-powder dispersers, liquid aerosol generators, or nebulizers (e.g., Pettit and Gombotz, TIBTECH 16:343 (1998); Patton et al., Adv. Drug Deliv. Rev. 35:235 (1999)). This approach is illustrated by the AERX diabetes management system, which is a hand-held electronic inhaler that delivers aerosolized insulin into the lungs. Studies have shown that proteins as large as 48,000 kDa have been delivered across skin at therapeutic concentrations with the aid of low-frequency ultrasound, which illustrates the feasibility of trascutaneous administration (Mitragotri et al., Science 269:850 (1995)). Transdermal delivery using electroporation provides another means to administer the scFc Type III Interferon fusion protein.

A composition comprising a scFc Type III Interferon fusion protein of the invention can be formulated according to known methods to prepare pharmaceutically useful compositions, whereby the therapeutic proteins are combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. See, for example, Gennaro (ed.), Remington's Pharmaceutical Sciences, 19th Edition (Mack Publishing Company 1995).

For purposes of therapy, a scFc Type III Interferon fusion protein of the present invention and a pharmaceutically acceptable carrier are administered to a patient in a therapeutically effective amount. A combination of a therapeutic scFc Type III Interferon fusion protein of the present invention and a pharmaceutically acceptable carrier is said to be administered in a “therapeutically effective amount” if the amount administered is physiologically significant. An agent is physiologically significant if its presence results in a detectable change in the physiology of a recipient patient. For example, an agent used to treat inflammation is physiologically significant if its presence alleviates the inflammatory response. Effective treatment may be assessed in a variety of ways. In one embodiment, effective treatment is determined by reduced inflammation. In other embodiments, effective treatment is marked by inhibition of inflammation. In still other embodiments, effective therapy is measured by increased well-being of the patient including such signs as weight gain, regained strength, decreased pain, thriving, and subjective indications from the patient of better health.

Determination of effective dosages in this context is typically based on animal model studies followed up by human clinical trials and is guided by determining effective dosages and administration protocols that significantly reduce the occurrence or severity of the patient disease or disorder in model subjects. Effective doses of the compositions of the present invention vary depending upon many different factors, including means of administration, target site, physiological state of the patient, whether the patient is human or an animal, other medications administered, whether treatment is prophylactic or therapeutic, as well as the specific activity of the composition itself and its ability to elicit the desired response in the individual. Usually, the patient is a human, but in some diseases, the patient can be a nonhuman mammal. Typically, dosage regimens are adjusted to provide an optimum therapeutic response, e.g., to optimize safety and efficacy. Accordingly, a therapeutically or prophylactically effective amount is also one in which any undesired collateral effects are outweighed by beneficial effects of inhibiting angiogenesis. For example, administration of an scFc molecule may have a dosage range from about 0.1 .micro.g to 100 mg/kg or 1 .micro.g/kg to about 50 mg/kg, and more usually 10 .micro.g to 5 mg/kg of the patient's body weight. In more specific embodiments, an effective amount of the agent is between about 1 .micro.g/kg and about 20 mg/kg, between about 10 .micro.g/kg and about 10 mg/kg, or between about 0.1 mg/kg and about 5 mg/kg. Dosages within these ranges can be achieved by single or multiple administrations, including, e.g., multiple administrations per day or daily, weekly, bi-weekly, or monthly administrations. For example, in certain variations, a regimen consists of an initial administration followed by multiple, subsequent administrations at weekly or bi-weekly intervals. Another regimen consists of an initial administration followed by multiple, subsequent administrations at monthly or bi-monthly intervals. Alternatively, administrations can be on an irregular basis as indicated by monitoring of a marker such as NK cell activity and/or clinical symptoms of the disease or disorder.

Dosage of the pharmaceutical composition may be varied by the attending clinician to maintain a desired concentration at a target site. For example, if an intravenous mode of delivery is selected, local concentration of the agent in the bloodstream at the target tissue may be between about 1-50 nanomoles of the composition per liter, sometimes between about 1.0 nanomole per liter and 10, 15, or 25 nanomoles per liter depending on the patient's status and projected measured response. Higher or lower concentrations may be selected based on the mode of delivery, e.g., trans-epidermal delivery versus delivery to a mucosal surface. Dosage should also be adjusted based on the release rate of the administered formulation, e.g., nasal spray versus powder, sustained release oral or injected particles, transdermal formulations, etc. To achieve the same serum concentration level, for example, slow-release particles with a release rate of 5 nanomolar (under standard conditions) would be administered at about twice the dosage of particles with a release rate of 10 nanomolar.

A composition comprising a scFc Type III Interferon fusion protein can be furnished in liquid form, in an aerosol, or in solid form. Liquid forms, are illustrated by injectable solutions, aerosols, droplets, topological solutions and oral suspensions. Exemplary solid forms include capsules, tablets, and controlled-release forms. The latter form is illustrated by miniosmotic pumps and implants. (See, e.g., Bremer et al., Pharm. Biotechnol. 10:239, 1997; Ranade, “Implants in Drug Delivery,” in Drug Delivery Systems 95-123 (Ranade and Hollinger, eds., CRC Press 1995); Bremer et al., “Protein Delivery with Infusion Pumps,” in Protein Delivery: Physical Systems 239-254 (Sanders and Hendren, eds., Plenum Press 1997); Yewey et al., “Delivery of Proteins from a Controlled Release Injectable Implant,” in Protein Delivery: Physical Systems 93-117 (Sanders and Hendren, eds., Plenum Press 1997).) Other solid forms include creams, pastes, other topological applications, and the like.

Liposomes provide one means to deliver therapeutic polypeptides to a patient intravenously, intraperitoneally, intrathecally, intramuscularly, subcutaneously, or via oral administration, inhalation, or intranasal administration. Liposomes are microscopic vesicles that consist of one or more lipid bilayers surrounding aqueous compartments (see, generally, Bakker-Woudenberg et al., Eur. J. Clin. Microbiol. Infect. Dis. 12 (Suppl. 1):S61 (1993), Kim, Drugs 46:618 (1993), and Ranade, “Site-Specific Drug Delivery Using Liposomes as Carriers,” in Drug Delivery Systems, Ranade and Hollinger (eds.), pages 3-24 (CRC Press 1995)). Liposomes are similar in composition to cellular membranes and as a result, liposomes can be administered safely and are biodegradable. Depending on the method of preparation, liposomes may be unilamellar or multilamellar, and liposomes can vary in size with diameters ranging from 0.02 .micro.m to greater than 10 .micro.m. A variety of agents can be encapsulated in liposomes: hydrophobic agents partition in the bilayers and hydrophilic agents partition within the inner aqueous space(s) (see, for example, Machy et al., Liposomes In Cell Biology And Pharmacology (John Libbey 1987), and Ostro et al., American J. Hosp. Pharm. 46:1576 (1989)). Moreover, it is possible to control the therapeutic availability of the encapsulated agent by varying liposome size, the number of bilayers, lipid composition, as well as the charge and surface characteristics of the liposomes.

Liposomes can adsorb to virtually any type of cell and then slowly release the encapsulated agent. Alternatively, an absorbed liposome may be endocytosed by cells that are phagocytic. Endocytosis is followed by intralysosomal degradation of liposomal lipids and release of the encapsulated agents (Scherphof et al., Ann. N.Y. Acad. Sci. 446:368 (1985)). After intravenous administration, small liposomes (0.1 to 1.0 .micro.m) are typically taken up by cells of the reticuloendothelial system, located principally in the liver and spleen, whereas liposomes larger than 3.0 .micro.m are deposited in the lung. This preferential uptake of smaller liposomes by the cells of the reticuloendothelial system has been used to deliver chemotherapeutic agents to macrophages and to tumors of the liver.

The reticuloendothelial system can be circumvented by several methods including saturation with large doses of liposome particles, or selective macrophage inactivation by pharmacological means (Claassen et al., Biochim. Biophys. Acta 802:428 (1984)). In addition, incorporation of glycolipid- or polyethelene glycol-derivatized phospholipids into liposome membranes has been shown to result in a significantly reduced uptake by the reticuloendothelial system (Allen et al., Biochim. Biophys. Acta 1068:133 (1991); Allen et al., Biochim. Biophys. Acta 1150:9 (1993)).

Liposomes can also be prepared to target particular cells or organs by varying phospholipid composition or by inserting receptors or ligands into the liposomes. For example, liposomes, prepared with a high content of a nonionic surfactant, have been used to target the liver (Hayakawa et al., Japanese Patent 04-244,018; Kato et al., Biol. Pharm. Bull. 16:960 (1993)). These formulations were prepared by mixing soybean phospatidylcholine, α-tocopherol, and ethoxylated hydrogenated castor oil (HCO-60) in methanol, concentrating the mixture under vacuum, and then reconstituting the mixture with water. A liposomal formulation of dipalmitoylphosphatidylcholine (DPPC) with a soybean-derived sterylglucoside mixture (SG) and cholesterol (Ch) has also been shown to target the liver (Shimizu et al., Biol. Pharm. Bull. 20:881 (1997)).

Alternatively, various targeting counter-receptors can be bound to the surface of the liposome, such as antibodies, antibody fragments, carbohydrates, vitamins, and transport proteins. For example, for targeting to the liver, liposomes can be modified with branched type galactosyllipid derivatives to target asialoglycoprotein (galactose) receptors, which are exclusively expressed on the surface of liver cells. (See Kato and Sugiyama, Crit. Rev. Ther. Drug Carrier Syst. 14:287, 1997; Murahashi et al., Biol. Pharm. Bull. 20:259, 1997.) In a more general approach to tissue targeting, target cells are prelabeled with biotinylated antibodies specific for a counter-receptor expressed by the target cell. (See Harasym et al., Adv. Drug Deliv. Rev. 32:99, 1998.) After plasma elimination of free antibody, streptavidin-conjugated liposomes are administered. In another approach, scFc Type III Interferon fusion proteins are directly attached to liposomes. (See Harasym et al., supra.)

Single Chain Type III Interferon fusion proteins can be encapsulated within liposomes using standard techniques of protein microencapsulation (see, for example, Anderson et al., Infect. Immun. 31:1099 (1981), Anderson et al., Cancer Res. 50:1853 (1990), and Cohen et al., Biochim Biophys. Acta 1063:95 (1991), Alving et al. “Preparation and Use of Liposomes in Immunological Studies,” in Liposome Technology, 2nd Edition, Vol. III, Gregoriadis (ed.), page 317 (CRC Press 1993), Wassef et al., Meth. Enzymol. 149:124 (1987)). As noted above, therapeutically useful liposomes may contain a variety of components. For example, liposomes may comprise lipid derivatives of poly(ethylene glycol) (Allen et al., Biochim. Biophys. Acta 1150:9 (1993)).

Degradable polymer microspheres have been designed to maintain high systemic levels of therapeutic proteins. Microspheres are prepared from degradable polymers such as poly(lactide-co-glycolide) (PLG), polyanhydrides, poly (ortho esters), nonbiodegradable ethylvinyl acetate polymers, in which proteins are entrapped in the polymer (Gombotz and Pettit, Bioconjugate Chem. 6:332 (1995); Ranade, “Role of Polymers in Drug Delivery,” in Drug Delivery Systems, Ranade and Hollinger (eds.), pages 51-93 (CRC Press 1995); Roskos and Maskiewicz, “Degradable Controlled Release Systems Useful for Protein Delivery,” in Protein Delivery: Physical Systems, Sanders and Hendren (eds.), pages 45-92 (Plenum Press 1997); Bartus et al., Science 281:1161 (1998); Putney and Burke, Nature Biotechnology 16:153 (1998); Putney, Curr. Opin. Chem. Biol. 2:548 (1998)). Polyethylene glycol (PEG)-coated nanospheres can also provide carriers for intravenous administration of therapeutic proteins (see, for example, Gref et al., Pharm. Biotechnol. 10:167 (1997)).

Other dosage forms can be devised by those skilled in the art, as shown, for example, by Ansel and Popovich, Pharmaceutical Dosage Forms and Drug Delivery Systems, 5th Edition (Lea & Febiger 1990), Gennaro (ed.), Remington's Pharmaceutical Sciences, 19th Edition (Mack Publishing Company 1995), and by Ranade and Hollinger, Drug Delivery Systems (CRC Press 1996).

As an illustration, pharmaceutical compositions may be supplied as a kit comprising a container that comprises a scFc Type III Interferons fusion protein of the present invention. The scFc Type III Interferon fusion proteins of the invention can be provided in the form of an injectable solution for single or multiple doses, or as a sterile powder that will be reconstituted before injection. Alternatively, such a kit can include a dry-powder disperser, liquid aerosol generator, or nebulizer for administration of the scFc Type III Interferon fusion protein. Such a kit may further comprise written information on indications and usage of the pharmaceutical composition.

A composition comprising a scFc Type III Interferon fusion protein of the invention can be furnished in liquid form, in an aerosol, or in solid form. Liquid forms, are illustrated by injectable solutions, aerosols, droplets, topological solutions and oral suspensions. Solid forms include capsules, tablets, and controlled-release forms. The latter form is illustrated by miniosmotic pumps and implants (Bremer et al., Pharm. Biotechnol. 10:239 (1997); Ranade, “Implants in Drug Delivery,” in Drug Delivery Systems, Ranade and Hollinger (eds.), pages 95-123 (CRC Press 1995); Bremer et al., “Protein Delivery with Infusion Pumps,” in Protein Delivery: Physical Systems, Sanders and Hendren (eds.), pages 239-254 (Plenum Press 1997); Yewey et al., “Delivery of Proteins from a Controlled Release Injectable Implant,” in Protein Delivery: Physical Systems, Sanders and Hendren (eds.), pages 93-117 (Plenum Press 1997)). Other solid forms include creams, pastes, other topological applications, and the like.

The present invention comprises compositions of scFc Type III Interferon fusion proteins that are either administered alone as a therapeutic, or in combination with other agents which are used to treat said condition, as well as methods for and therapeutic uses of the scFc Type III Interferon fusion protein itself. Such compositions can further comprise a pharmaceutical acceptable carrier. The pharmaceutical acceptable carrier can be a conventional organic or inorganic carrier. Examples of carriers include water, buffer solution, alcohol, propylene glycol, macrogol, sesame oil, corn oil, and the like.

The complete disclosure of all patents, patent applications, and publications, and electronically available material (e.g., GenBank amino acid and nucleotide sequence submissions) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. An isolated fusion protein comprising from the amino-terminus to the carboxy-terminus a polypeptide having at least 90% or 95% sequence identity to amino acid residues 1-181 of SEQ ID NO:1, a linker polypeptide and a scFc polypeptide comprising at least two Fc monomers and at least one linker, wherein the first Fc monomer comprises a CH2 domain and a CH3 domain and the second Fc monomer comprises a CH2 domain and a CH3 domain.

2. The isolated fusion protein of claim 1, wherein said first Fc monomer and said second Fc monomer are arranged in an amino to carboxyl order selected from the group consisting of:
a) Hinge-CH2-CH3-linker-Hinge-CH2-CH3;
b) Hinge-CH2-CH3-linker-CH2-CH3;
c) Hinge-CH2-linker-Hinge-CH2-CH3-linker-CH3;
d) Hinge-CH2-linker-CH2-CH3-linker-CH3;
e) linker-CH2-CH3-linker-CH2-CH3; and
f) CH2-linker-CH2-CH3-linker-CH3.

3. The isolated fusion protein of claim 1, wherein the first and second Fc monomers have no effector function or have a substantially reduced effector function.

4. The isolated fusion protein of claim 1, wherein the first and second Fc monomers are SEQ ID NO:11, SEQ ID NO:12, SEQ ID NO:13 or SEQ ID NO:14.

5. The isolated fusion protein of claim 1, further comprising from the carboxy-terminus of the fusion protein a second linker and a second polypeptide, wherein the second polypeptide has at least 90% or at least 95% sequence identity to amino acid residues 1-181 of SEQ ID NO:1.

6. The isolated fusion protein of claim 5, further comprising from the amino-terminus of the fusion protein a third linker and a third polypeptide, wherein the third polypeptide has at least 90% or at least 95% sequence identity to amino acid residues 1-181 of SEQ ID NO:1.

7. The isolated fusion protein of claim 6, further comprising from the carboxy-terminus of the fusion protein a fourth linker and a fourth polypeptide, wherein the fourth polypeptide has at least 90% or at least 95% sequence identity to amino acid residues 1-181 of SEQ ID NO:1.

8. The isolated fusion protein of claim 7, wherein the linker, second linker, third linker or fourth linker is (Gly₄Ser)ₙ, wherein n is 1-10.

9. The isolated fusion protein of claim 7, wherein the linker, second linker, third linker or fourth linker is selected from the group consisting of SEQ ID NO:4, SEQ ID NO:5, SEQ ID NO:6, SEQ ID NO:7, SEQ ID NO:8, SEQ ID NO:9 and SEQ ID NO:10.

10. A composition comprising the fusion protein according to any one of claim 1, 5, 6 or 7 and a pharmaceutically acceptable carrier.

11. An isolated polynucleotide encoding the fusion protein according to any one of claim 1, 5, 6 or 7.

12. An expression vector comprising the following operably linked elements:
(a) transcription promoter;
(b) a DNA segment encoding the fusion protein according to any one of claims 1, 5, 6 or 7; and
(c) transcription terminator.

13. A cultured cell comprising the expression vector of claim 12.

14. A method of producing a fusion protein comprising:
culturing the cell according to claim 13 under conditions wherein the fusion protein is expressed from the expression vector and recovering the expressed fusion protein.

15. A method of treating a patient having a viral infection comprising administering to the patient a therapeutically effective amount of the composition of claim 10, wherein after administration of the composition the viral load is reduced or viral replication is inhibited, wherein the viral infection is hepatitis B or hepatitis C.


SEQUENCE LISTING (25 sequences)

SEQ ID NO: 1 - protein, 181 aa, Homo sapiens
GPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 2 - protein, 175 aa, Homo sapiens
VPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 3 - protein, 175 aa, Homo sapiens
VPVARLRGALPDARGCHIAQFKSLSPQELQAFKRAKDALEESLLLKDCKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 4 - protein, 15 aa, Artificial Sequence
  linker
GGGGSGGGGSGGGGS

SEQ ID NO: 5 - protein, 17 aa, Artificial Sequence
  linker
GXXXXXGXXXXXGXXXX

SEQ ID NO: 6 - protein, 11 aa, Artificial Sequence
  linker
GGGXGGGXGGG

SEQ ID NO: 7 - protein, 25 aa, Artificial Sequence
  linker
GXXXXXXXXXXXCGXXXXXXXXXXX

SEQ ID NO: 8 - protein, 11 aa, Artificial Sequence
  linker
GGGGGCGGGGG

SEQ ID NO: 9 - protein, 41 aa, Artificial Sequence
  linker
GGGGSGGGGSGGGGSGGGGSGGGGSGGGGSGGGGSGGGGSG

SEQ ID NO: 10 - protein, 34 aa, Artificial Sequence
  linker
KPTTTPAPRPPTPAPTIASQPLSLRPEASRPAAG

SEQ ID NO: 11 - protein, 232 aa, Homo sapiens
EPRSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 232 aa, Homo sapiens
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 231 aa, Homo sapiens
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 223 aa, Homo sapiens
TCPPCPAPELLGGPSVFLFPPKPKDTLMISRTPEVTCVVVDVSHEDPEVKFNWYVDGVEVHNAKTKPREEQYESTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 489 bp, Homo sapiens
  CDS (1)..(489)
cgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttctattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaatacgttgctgatggtaacctgtctctgcgtacctctacccatccggaatctacc

SEQ ID NO: 17 - protein, 163 aa, Homo sapiens
RFKSLSPQELASFKKARDALEESLKLKNWSCSSPVFPGNWDLRLLQVRERPVALEAELALTLKVLEAAAGPALEDVLDQPLHTLHHILSQLQASIQPQPTAGPRPRGRLHHWLHRLQEAPKKESAGCLEASVTFNLFRLLTRDLKYVADGNLSLRTSTHPEST

SEQ ID NO: 18 - DNA, 483 bp, Homo sapiens
  CDS (1)..(483)
aaaccaaccaccactggtaaaggttgccacatcggtcgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttgcattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaatacgttgctgat

SEQ ID NO: 19 - protein, 161 aa, Homo sapiens
KPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVAD

SEQ ID NO: 20 - DNA, 471 bp, Homo sapiens
  CDS (1)..(471)
aaaccaaccaccactggtaaaggttgccacatcggtcgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttgcattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaa

SEQ ID NO: 21 - protein, 157 aa, Homo sapiens
KPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLK

SEQ ID NO: 22 - DNA, 447 bp, Homo sapiens
  CDS (1)..(447)
cgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttctattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaatacgttgctgat

SEQ ID NO: 23 - protein, 149 aa, Homo sapiens
RFKSLSPQELASFKKARDALEESLKLKNWSCSSPVFPGNWDLRLLQVRERPVALEAELALTLKVLEAAAGPALEDVLDQPLHTLHHILSQLQASIQPQPTAGPRPRGRLHHWLHRLQEAPKKESAGCLEASVTFNLFRLLTRDLKYVAD

SEQ ID NO: 24 - DNA, 435 bp, Homo sapiens
  CDS (1)..(435)
cgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttctattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaa

SEQ ID NO: 25 - protein, 145 aa, Homo sapiens
RFKSLSPQELASFKKARDALEESLKLKNWSCSSPVFPGNWDLRLLQVRERPVALEAELALTLKVLEAAAGPALEDVLDQPLHTLHHILSQLQASIQPQPTAGPRPRGRLHHWLHRLQEAPKKESAGCLEASVTFNLFRLLTRDLK